(19) United States
(12) Patent Application Publication
(10) Pub. No.: US 2019/0274687 A1
(43) Pub. Date: Sep. 12, 2019

(54) MAGNETIC ANASTOMOSIS DEVICES

(71) Applicant: Neurotronic, Inc., Plymouth, MN (US)

(72) Inventors: Lixiao Wang, Henderson, NV (US); John J. Chen, Plymouth, MN (US); Yongxing Zhang, Irvine, CA (US); Craig C. Belton, Prior Lake, MN (US); Shannon R. Stroud, Osseo, MN (US); Richard Cornelius, Wayzata, MN (US); Scott Naisbitt, Orono, MN (US)

(21) Appl. No.: 16/334,813

(22) PCT Filed: Sep. 20, 2017
(86) PCT No.: PCT/US17/52498

§ 371 Date: Mar. 20, 2019

Related U.S. Application Data
(60) Provisional application No. 62/397,251, filed on Sep. 20, 2016.
(60) Provisional application No. 62/434,817, filed on Dec. 15, 2016.
(60) Provisional application No. 62/501,251, filed on May 4, 2017.

(51) Int. Cl. A61B 17/11

(52) U.S. Cl. CPC A61B 17/1114; A61B 2017/00876

(57) ABSTRACT

Embodiments of the present invention provide a magnetic anastomosis device (MAD), a delivery catheter for the MAD, and a method for treating at least one of diabetes, obesity, digestive disease, and non-alcoholic fatty liver disease. In embodiments of the present invention, a pair of the MADs are delivered in body lumen by endoscopic and/or laparoscopic techniques. An anastomosis between two adjacent body lumens is formed by compression of the pair of MADs followed by necrosis and regeneration of the lumen tissue. The bodily fluids are then redirected through the formed anastomosis to relieve disease symptoms.

### CROSS-REFERENCE TO RELATED APPLICATIONS

This application claims the benefit of priority to U.S. Provisional Patent Application Ser. No. 62/397,251 filed Sep. 20, 2016, U.S. Provisional Patent Application Ser. No. 62/434,817 filed Dec. 15, 2016, and to U.S. Provisional Patent Application Ser. No. 62/501,251 filed May 4, 2017, the disclosures of which are incorporated herein in their entirety by reference.

### BACKGROUND

Diabetes is a metabolic condition, or combination of conditions, where an individual experiences high concentrations of blood glucose. The condition is caused either by insufficient production of insulin within the body or by failure of cells to respond properly to insulin. HbA1c (A1c) value is clinically used for diabetes diagnosis. HbA1c refers to glycated hemoglobin, which identifies average plasma glucose concentration. In human, normal HbA1c<6.0%, prediabetes HbA1c6.0% to 6.4%, diabetes>6.5%.

Diabetes is one of the leading causes of death and disability in the United States and in other developed countries. It is associated with long-term complications that affect almost every part of the body. It has been linked, for instance, to blindness, heart and blood vessel disease, stroke, kidney failure, amputations, and nerve damage.

Within the United States, diabetes affects approximately 8 percent of the population and has resulted in costs that approach $250 billion.

Diabetes is typically classified as either type 1 (also referred to as insulin-dependent diabetes or juvenile diabetes), wherein the patient fails to produce sufficient insulin, type 2 (also referred to as non-insulin-dependent diabetes, adult-onset diabetes, or obesity-related diabetes), wherein the patient fails to respond properly to insulin, or gestational diabetes, a condition which develops late in pregnant women.

Type 2 diabetes is the most common form of diabetes, accounting for 90% to 95% of overall cases. It is generally associated with older age, obesity, family history, previous history with gestational diabetes, and physical inactivity. It is also more prevalent in certain ethnicities. Type 2 diabetes is also referred to as insulin-resistant diabetes, as the pancreas typically produces sufficient amounts of insulin, but the body fails to respond properly it. Symptoms include fatigue, frequent urination, increased thirst and hunger, weight loss, blurred vision, and slow healing of wounds or sores.

Obesity is another significant health concern, particularly in the developed world. It is a complex, multifactorial and chronic condition characterized by excess body fat, which results from an imbalance between energy expenditure and caloric intake. Although the causes of this imbalance are not completely understood, genetic and/or acquired physiologic events and environmental factors are thought to contribute. The adverse health effects associated with obesity, and more specifically morbid obesity, have become well-established in recent years. Such adverse effects include, but are not limited to, cardiovascular disease, diabetes, high blood pressure, arthritis, and sleep apnea. Generally, as a patient's body mass index (BMI) rises, the likelihood of suffering the adverse effects linked to obesity also rises.

Nonalcoholic fatty liver disease is another health concern, which occurs when your liver has trouble breaking down fats, causing fat to build up in your liver tissue of people who drink little or no alcohol. Nonalcoholic fatty liver disease is common and, for most people, causes no signs and symptoms and no complications. But in some people with nonalcoholic fatty liver disease, the fat that accumulates can cause inflammation and scarring in the liver. This more serious form of nonalcoholic fatty liver disease is sometimes called nonalcoholic steatohepatitis. At its most severe, nonalcoholic fatty liver disease can progress to liver failure.

A magnetic anastomosis device (MAD) have been reported in USSR Inventors' Certificate No. 1,179,978, USSR Inventors' Certificate No. 736,966, U.S. Pat. Nos. 5,690,656, 8,118,821, 8,142,454, 8,870,899, and 9,421,015. A MAD can be used to create a channel or anastomosis between two body lumens or organs for the purpose of redirecting bodily fluids. The body lumens and organs includes stomach, small and large intestines, liver, heart, lung, colon, duodenum, jejunum, ileum, biliary duct, gallbladder, urological lumen, veins, arteries, and esophagus. The MAD creates a compressive anastomosis between two body lumens or organs. Because of the strong compression, the tissue trapped between the two MADs is cut off from its blood supply. Under these conditions, the tissue becomes necrotic and degenerates, and at the same time, new tissue grows around points of compression, e.g., on the edges of the coupling. When the coupling is removed, a healed anastomosis between the two tissues is formed. Very limited success in human trials has been obtained from these MADs. There is no commercial MAD available on the market today. Improvements of the safety and efficacy of these devices are needed. The delivery of these devices deep in the gastrointestinal tracts remains challenging.

### SUMMARY OF THE INVENTION

Embodiments of the present invention relate to a MAD, MAD delivery catheters, and methods of treatment for diabetes, obesity, nonalcoholic fatty liver disease, digestive diseases, cancers, and tumors. The delivery catheters can include a combination of a MAD and their delivery system. The deployable magnetic devices can be used for creating anastomoses between two body lumens or organs for the purpose of redirecting/diversion of body fluids. The body lumens and organs can include small and large intestines, colon, duodenum, jejunum, ileum, biliary duct, gallbladder, urological lumen, veins, arteries, esophagus or a combination thereof. The methods can involve delivery of the magnetic devices to targeted body lumens in the human body by endoscopic and/or laparoscopic techniques. The methods can include the creation of an anastomoses between two body lumen and/or organs with MAD and its delivery systems using endoscopic and/or laparoscopic techniques. The inventions improve safety and efficacy of the magnetic devices and their delivery system including minimum invasive or non-invasive procedure and ready to mate MAD rings that have no plane face orientation requirement to pair them together.

In one embodiment of the present invention the MADs in human body for treatment of diabetes, obesity and non-alcohol fatty liver disease comprises (1) at least one pair of MAD rings; (2) at least two layer tissues of the body lumen to be joined at the beginning of anastomosis formation and to necrose over time; and (3) reducing Glycated Hemoglobin A1c (HGBA1C or HbA1c or A1C), plasma glucose concentration and body weight over time.

wherein the two layers of the body lumen are in between the two MAD rings; wherein the MAD ring is enclosed in a polymer heat shrink tube (PHST) and the polymer heat shrink tube is flexible and conformable so that the MAD forms a ring shape from a linear shape; wherein the tissues applicable to such a treatment include nonvascular lumens, digestive lumens, stomach, esophagus, bile duct, duodenum, jejunum, ileum, cecum, colon, cancers, and tumors.

In one embodiment of the present invention the MAD can be delivered in a low profile, linear configuration using a catheter, endoscope, endoscopic overtube, or laparoscope. Some examples of endoscopes used in human intestines include a work channel having a size (e.g., diameter) in the range of 2.8 mm to 3.8 mm. The linear crossing profiles of invented MADs are compatible with these scopes. Upon delivery, the MAD can be formed into the ring shape by guiding elements in small intestines such as duodenum, jejunum, ileum, and can be paired to a second MAD ring to join jejunum and ileum tissues or duodenum and ileum tissues by a pull-back method. The blood supply through the tissues compressed in-between the mated surfaces of two MAD rings is cut off. The ischemic tissues can be allowed to necrose over time and fall away through the digestive track to form an anastomosis.

The anastomosis can allow a portion of the digestive fluids and food to bypass a portion of the intestinal path through the MAD while the partially bypassed jejunum, duodenum and ileum tissue continues to function in its native capacity with the portion of the digestive fluids and food which do not pass through the anastomosis. By selecting the sizes of the MAD, a physician can control the amount of diversion based on needs of the patients to control diseases, such as diabetes and obesity.

In another embodiment of the present invention the MAD rings can be used to join small intestines and large intestine such as colon to jejunum, colon to ileum colon to duodenum, cecum to jejunum, cecum to ileum or cecum to duodenum.

Embodiments of the MAD in the present invention include a tube, such as a heat shrink polymeric tube (HSPT), and a plurality of magnets, wherein the magnets are assembled in the tube that acts as an enclosure; the magnets are inserted with proper pole directions into the heat shrink tube; also the magnets can be inserted with the proper alignment of their special geometric features into one direction in the heat shrink tube for the lowest possible crossing profile; then heating is added to shrink the polymeric tube over the assembled magnets; the magnets assembled with proper pole directions are then fixed in the polymeric tube. The MAD is generally ring- or disc-shaped with a hole in the center. The outer diameter (D) of the ring is in the range of 5 mm to 50 mm, or 10 mm to 35 mm. The width of the MAD is in the range of 0.5 mm to 10 mm, or preferably, 1 mm to 5 mm. The thicknesses of the MAD is in the range of 0.25 mm to 10 mm, or 1 mm to 4 mm. The magnets in the MAD are discs, cylinders, circles, oblongs, ovals, squares, blocks, cubes, hexagonal, octagonal, and trapezoidal shapes with/without holes, or with modified features such as a chamfer, bevel, fillet, and the like. The magnets in the MAD are coated with metals or polymers or the combination of both in layers for protection and biocompatibility. The diameter or dimension d of the magnets are the same as the width of the MAD. The thickness of the magnets plus the double wall thickness of the heat shrink tubes are equal to the thickness of the MAD. The number of the magnets in the MAD is in the range of 1-40, 8-30, 10-30, 12-30, 14-30, or 16-26. The number can be an even number. When the magnets are magnetized thru their thickness direction are arranged in alternating polarities pattern to each other (e.g., NSNSNS pattern), the even number gives both ends having opposite magnetic pole directions, which are attractive to each other when formed into a ring. If the magnets are disc or cylinder the outer diameter (D) of the MADS can be estimated by the product of the number of the magnets (n) multiplied by the diameter (d) of the magnets divided by π and then adding the diameter (d) of the magnets. D=n d/π+d. For example, the diameter (D) of MAD is 19.35 mm if n is 16 and d is 3.175 mm; the diameter (D) of MAD is 13.29 mm if n is 10 and d is 3.175 mm; the diameter (D) of MAD is 18.72 mm if n is 20 and d is 2.54 mm. All these numbers are for illustration only since they are theoretical calculations. In the case of an actual device the calculated results above have not included any gap between the magnet discs and the wall thickness of the heat shrink sheath wrapping around the discs.

For magnet mass, magnetic strength, MAD ring size (outer diameter), MAD linear crossing profile (endoscope compatibility) and ease to mate between the first MAD ring and the second MAD ring, suitable numbers of magnets in the ring of current invention are 1-40, 8-30, 10-30, 12-30, 14-30, or 16-26; and suitable attraction forces of these rings are 0.7 pounds to 4.0 pounds as measured with a gap distance of about 1 mm between the rings for applications jointing two segments of the intestine. Stronger attractive forces would be desirable for applications joining thicker walled body lumens (e.g., the stomach).

In one embodiment, the polymer heat shrink tube (PHST) includes a polyester, a polyamide, a polyether-amide block copolymer (e.g., Pebax), a polyolefin, a polyolefin derivative, a polyurethane, a poly(vinyl chloride), a polytetrafluoroethylene (PTFE), a fluorinated ethylene propylene (FEP), a perfluoroalkoxy polymer (PFA), or a combination thereof. The shrinking temperature of the heat shrink tubes can depend on the polymer materials and can be in the range of 45° C. to 300° C. Low shrinking temperature materials can be desirable since temperature can have impact on the magnetic strength of the magnets (degrading); the low heat shrinking temperature can be in a range of from about 60° C. to about 130° C., about 70° C. to about 120° C., or less than, equal to, or greater than about 60° C., 65° C., 70° C., 75° C., 80 ° C., 85° C., 90° C., 95° C., 100° C., 105° C., 110° C., 115° C., 120° C., 125° C., or about 130° C. This can help to minimize the risk of degrading the magnet strength. The size of the heat shrink tubes can be slightly larger than the sizes of the magnets in the MADs. More specifically, the circumference of the heat shrink tube inside diameter (C_{ID}) is equal to or slightly larger than the perimeter (P_{cs}) of the cross section area of the magnets in the alignment they will reside in the tubing in their final assembled state. The ratio of C_{ID}/P_{cs} can be ≥1 and ≤2. The shrinking ratio can be in the range of 1.1:1 to 4:1 and should generally allow for the PHST to shrink to a relatively snug fit over the magnets. In some examples, the heat shrink tube selection should match the temperature grade of the magnets, so the heat shrink process will not degrade the magnetic strength of the MAD rings. Heat shrink tube is also required to have shape retention feature; so it can be re-circularized from linear form during delivery to the ring.

Embodiments of MAD delivery catheters in the present invention include MAD, pushing tubes, guiding elements and introducer. The pushing tubes can be solid or hollow, for example, metal wires, such as stainless steel wires, nitinol wires, or hypotubes or plastic tubing, such as stainless steel hypotube, nitinol hypotube, layered polymer tubes, braided tube, or plastic tubing. Plastic tubing can include unfilled high density polyethylene (HDPE), or filled polyethylene such as barium sulfate-filled HDPE which is radiopaque, or other polymers. The guiding elements can be single or multiple filaments of polyesters, polyamides, polypropylene, polyethylene (e.g., ultra-high molecular weight polyethylene) or fluoropolymers, or metal wire such as steel wire and nitinol wire. The completed assembly of the MAD delivery catheter can include the MAD in linear deployable shape in the distal end, followed by the pushing tube. The guiding elements can be attached to the MAD in the distal end and extended through or along the pushing tube to the proximal end of the MAD delivery catheter. The guiding elements can encircle the outside of the MAD diameter and inside of the heat shrink tube. The guiding elements can also be connected near the ends of the MAD. Optionally the MAD system can be enclosed in a delivery sheath and the MAD can be preloaded at the distal end of the sheath. The sheath can be a short introducer to connect MDA with the pushing rod. The length of short introducer can be about 12 inches or 6 inches. The MAD can be fully or partially covered by the introducer. The materials of the introducer are thermoplastics, preferably, modified thermoplastic that provide low friction to release the MAD. Suitable materials are PTFE modified, FEP, silicone modified, and PebaSlix (Duke Empirical, Santa Cruz, Calif.). The MAD can be manipulated to place it in the body lumen preselected location and direction by the guiding elements.

In some embodiments, the pushing tube includes two different tubes coaxially arranged one inside of another. Any suitable thermoplastic or its thermoplastic elastomer can be used for the outside layer material, such as polyamide, polyester, polyethylene, polypropylene and other engineering thermoplastic materials. The inside tube includes polytetrafluoroethylene (PTFE) or other fluoropolymers that reduce friction and drag for guiding elements operation.

In one embodiment, the MAD delivery catheter has an atraumatic soft tip at the distal end of the pushing tube which has shore D hardness less than 55D, less than 50D, or less than 40D.

In one embodiment, the removable attachment features can be directly fixed on to the end pieces of MAD magnets, such as on the open ends, which can enable ring formation after delivery. The attachment feature can include a stainless steel wire with a small eyelet or arc shape loop to hold delivery lines and allow the delivery line withdrawn easily via rolling/sliding through the hole. In addition to metal wire, the attachment can include plastic parts such as injection molded plastic parts with micro-injection molding technology.

In one embodiment, the delivery guiding element lines for ring formation can be attached directly on to the heat shrink tube at or near the two open end pieces of the magnets. In some embodiments, the removable attachments can be broken away at the heat shrink tube site after the two MAD devices have magnetically joined across the body lumen walls. As an embodiment, the breakaway can be achieved with a controlled amount of force with the feature of enlarged end diameter of guiding element lines, which is larger than the precisely controlled hole size in the sheath where the lines pass through. The enlarged ends of the lines require a controlled amount of force to be pulled through the holes in the sheath to separate the pushing tubes and lines from the magnet ring and sheath. The suitable force is 0.25 to 5 lbs. or 0.5 to 3 lbs. The enlarged ends of the lines can be achieved with knots or molded on beads as two possible examples. In another embodiment, a single guiding element line may pass through the heat-shrink tube of the proximal magnet and be attached with a breakaway knot to the heat-shrink tube at the distal end, so that as it is retracted it pulls proximal and distal ends together to circularize the magnet ring. As another exemplary embodiment, the force to separate the ring and sheath from the guiding element lines can be controlled by having the guiding element line connected to the sheath by a loop through two small holes very closely spaced apart in the sheath wall. The line can be separated by pulling the loop until it causes the material in the sheath wall between these holes to tear. This separation force can be controlled by the spacing of the two holes.

In another embodiment, the guiding element lines are connected to the MAD through holes in the side of the heat shrink tubing covering the ring which is covering the outside thickness of the magnet ring. When connected in this way to rings which have the rings formed from magnets having alternating polarities, this yields a MAD system which is symmetric with the top surface of the ring being functionally equivalent to the bottom surface of the ring. This allows the pair of rings to be mated between adjacent body lumens without the need to control which face of the ring is directed towards the ring in the other body lumen.

In one embodiment, the MAD delivery catheter can be delivered with assisting devices. The assisting devices can reduce endoscope loops inside of the body during delivery or enable a standard endoscope to be advanced or positioned in the deep small and large intestine. The combination of the endoscope and assisting device can enhance the reach of the targeted sites deeper in body lumen such as colon, duodenum, jejunum, and ileum beyond the ligament of Treitz or ileocecal junction. The assisting devices match with the endoscope for appropriate internal diameter or outer diameter and length. The assisting devices can include an overtube, single balloon overtube, a double balloon overtube, a spiral overtube, a motorized spiral overtube, a G-EYE endoscope system, and a NaviAid balloon system.

In one embodiment, the MAD delivery catheter can be delivered with the assisting devices. The assisting device is one of overtube, single balloon overtube, double balloon overtube, spiral overtube, and motorized spiral overtube. These overtubes are accessory to an endoscope that can reduce endoscope loops inside of the body during delivery and enable a standard endoscope to be advanced or positioned in the small and large intestine. The combination of the endoscope and overtube can enhance the reach of the targeted sites deeper in body lumen such as colon, duodenum, jejunum, and ileum. The overtube matches with the endoscope for appropriate internal diameter and length. In some embodiments, the Reach Overtube by US endoscope, Mentor, Ohio, is used with a standard endoscope of outside diameter of 11.5 mm. The single balloon enteroscope system from Olympus is an example of single balloon overtubes. Another example of the overtubes is the double balloon endoscopy system from FUUJIFILM. G-EYE endoscope system and NaviAid Balloon system from Smart Medical are another kind of the assisting devices that fit into work channel of the scopes.

In one embodiment, the method of delivering MAD in body lumen includes: 1) inserting and advancing the first endoscope with the first assisting devices transorally into stomach and duodenum to or beyond targeted site, such as jejunum; 2) inserting and advancing the second endoscope or colonoscope transanally into colon or ileum with the second assisting device to or beyond targeted site, such as ileum; 3) identifying the point on both endoscopes that are in close proximity; 4) delivering the MAD from the working channels of the endoscopes; 5) transforming the MAD from linear to circular shape; 6) manipulating or withdrawing the endoscopes to the previously identified point of proximity and mating two MAD rings with each other from two separated lumens/organs; 7) using the tip of the delivery system to support the side of the MAD ring while pulling the guide element lines to separate the lines from the MAD rings, 8) withdrawing the MAD delivery systems into the endoscopes and withdrawing the endoscopes from the body; and 9) forming anastomosis over time. The first endoscope is different from the second endoscope. The first assisting device can be the same or different from the second assisting device. The assisting devices include overtube, single balloon overtube, double balloon overtube, spiral overtube, motorized spiral overtube, G-EYE endoscope system, and NaviAid balloon system. The first and the second assisting device can be NaviAid balloon system in one embodiment. The first assisting device is one of overtubes and the second assisting device is NaviAid balloon system in another embodiment. The pairs of the first and the second assisting devices are single balloon overtube and double balloon overtube, two overtubes, two double balloon systems, and two single balloon systems, one single balloon system and one double balloon system, two G-EYE endoscope systems, two NaviAid Balloon systems, and One G-EYE endoscope system and one NaviAid Balloon system.

In an embodiment, the endoscope can reach deep in the duodenum, jejunum, ileum or colon with the assistance of endoscopic balloon catheters, such as NaviAid Balloon catheter (Smart Medical Systems Ltd., Israel). The role of the balloon catheter is to facilitate advancement of a standard endoscope into the small and large intestine. In one embodiment, the method of delivering MAD in body lumen including: 1) inserting the first endoscope transorally into stomach and duodenum, passing ligament treitz; 2) inserting a balloon catheter such as NaviAid AB (40 mm balloon) in the working channel of the endoscope and advancing beyond the scope distal tip; 3) moving the balloon catheter into deep lumen such as jejunum; 4) inflating the balloon catheter to fix it at a position as an anchoring device for endoscope to advance or for reducing stomach loops; 5) moving the endoscope over the balloon catheter to deep lumen, such as jejunum until reaching the anchoring balloon catheter; 6) repeating step 3), 4) and 5) until reaching beyond the targeted site; 7) withdrawing the balloon catheter from the scope; 8) inserting the second endoscope or colonoscope transanally into colon or ileum; 9) inserting a balloon catheter such as NaviAid balloon catheter in the working channel of the endoscope and advancing beyond the endoscope distal tip; 10) moving the balloon catheter into deep lumen such as the ileum; 11) inflating the balloon catheter to fix it at a position as an anchoring device for the endoscope to advance; 12) moving the endoscope over the balloon catheter to deep lumen, such as ileum until reaching the anchoring balloon catheter; 13) repeating the last three steps until reaching beyond the targeted site; 14) using orthogonal fluoroscopic views, identify the target mating site by locating the points on both endoscope shaft loops that are in close proximity to each other; 15) withdrawing the balloon catheter; 16) delivering the MADs from the working channels of the endoscopes in a linear form; 17) transforming the MADs from linear to circular shape; 19) adjusting or moving back the tips of the endoscopes to line up the two MADs to the pre-specified target mating site; 20) mating two MAD rings with each other from two separated lumens/organs; 21) using X-ray image to confirm the mating; 22) detaching or separating the guiding element lines from the mated MADs; 23) withdrawing the MAD delivery systems from the endoscopes; 24) withdrawing the endoscopes carefully without touching the deployed MADs; 25) forming anastomosis over time by pressure necrosis, 26) allowing the necrotized tissue together with the two MADs to flow out of the body through the digestive tract.

In one embodiment, the method of delivering MAD in body lumen includes: 1) sliding the first endoscope into an overtube until its proximal end; 2) inserting the combined devices transorally into stomach and duodenum; 3) pulling the endoscope back to reduce endoscope loops in stomach; 4) advancing the overtube as far as desired to minimize looping in the stomach or intestine; 5) advancing the endoscope to or beyond targeted site, such as jejunum; 6) inserting the second endoscope or colonoscope transanally into colon or ileum; 7) inserting a balloon catheter such as NaviAid balloon catheter in the working channel of the endoscope and advancing beyond the scope distal tip; 8) moving the balloon catheter into deep lumen such as ileum; 9) inflating the balloon catheter to fix it at a position as an anchoring device for the endoscope to advance; 10) moving the endoscope over the balloon catheter to deep lumen, such as ileum, until reaching the anchoring balloon catheter; 11) repeating the last three steps until reaching to or beyond the targeted site; 12) withdrawing the balloon catheter from the scope; 13) identifying the point on both endoscopes that are in close proximity; 14) delivering the MAD from the working channels of the endoscopes; 15) transforming the MAD from linear to circular shape; 16) manipulating or withdrawing the endoscopes to the previously identified point of proximity and mating two MAD rings with each other from two separated lumens/organs; 17) Using the tip of the delivery system to support the side of the MAD ring while pulling the guide element lines to separate the lines from the MAD rings, 18) withdrawing the MAD delivery systems into the endoscopes and withdrawing the endoscopes from the body; and 19) forming anastomosis over time.

In one embodiment, the present invention provides a method for delivering the MAD to a body lumen, the method including: inserting a MAD catheter and advancing it to the target site in the body lumen; releasing the MAD to the body lumen; inserting the second MAD catheter and advancing it to the target site to be joined in a body lumen; releasing the second MAD to the body lumen; manipulating guiding elements to orientate and place both of the MADs in a location and orientation to couple (e.g., to mate); withdrawing the MAD delivery systems from the body lumen (e.g., after detaching the guiding lines). The MAD can include: at least ten, twelve, fourteen, or sixteen magnets; and a flexible polymeric tube, wherein a plurality of the magnets are assembled and fixed in the right direction and the right place in the polymeric tube upon heating, which is in the ring form before delivery; and wherein the polymeric tube is flexible to allow the MAD to direct into a geometric shape (e.g., from ring to linear form and back to ring shape).

In various embodiments, the present invention provides a method for delivering the MAD to a body lumen, including two endoscopes advancing to the target site. For example, the method can include inserting an endoscope and advancing it to the target site in the first body lumen from one direction, and inserting second endoscope and advancing to the target site in the second body lumen from another direction. In some cases, the tips of the two endoscopes come into close proximity during advancement or manipulation. Then, two scope tips are adjacent to each other, the MADs are delivered from both direction and mated. However, in other cases, it is very challenging to bring tips into close proximity during advancement. But as the endoscopes are advanced, the scope shaft loops of the two endoscopes press against each other, and a point on the scope shafts may be identified at which the tips will be in proximity during withdrawal, which is an anatomically convenient site for mating. In various embodiments, the present invention provides a method for delivering the MAD to a body lumen, including two endoscopes advancing beyond the target site. For example, the method can include inserting an endoscope and advancing it beyond the target site in the first body lumen from one direction, and inserting second endoscope and advancing it beyond the target site in the second body lumen from another direction. An anatomically convenient mating site may then be identified as the point on the shafts at which the scope loops of the first and second endoscopes are proximate in two orthogonal planes by fluoroscopy. The MADs may then be delivered from both directions and mated after or while pulling back the endoscopes and the MADs to the desired target anastomosis site. This pull-back method has several advantages, as it precludes inadvertent capture of other tissues between mated magnets and shortens the procedure time. It also allows the primary manipulation of the rings to be drawing them back through the body lumen which makes control of the rings alignment easier than when trying to advance them forward down the body lumen.

In the embodiments of the present invention the methods to treat the diseases of diabetes and obesity comprises 1) inserting and advancing the first endoscope with the first assisting devices transorally into stomach and duodenum to or beyond targeted site, such as jejunum; 2) inserting and advancing the second endoscope or colonoscope transanally into colon or ileum with the second assisting device to or beyond targeted site, such as ileum; 3) identifying the point on both endoscopes that are in close proximity; 4) delivering the MAD from the working channels of the endoscopes; 5) transforming the MAD from linear to circular shape; 6) manipulating or withdrawing the endoscopes to the previously identified point of proximity and mating two MAD rings with each other from two separated lumens/organs; 7) using the tip of the delivery system to support the side of the MAD ring while pulling the guide element lines to separate the lines from the MAD rings, 8) withdrawing the MAD delivery systems into the endoscopes and withdrawing the endoscopes from the body; and 9) forming anastomosis over time; 10) reducing Glycated Hemoglobin A1c(HGBA1C or HbA1c or A1C), plasma glucose concentration and body weight over time. The first endoscope is different from the second endoscope. The first assisting device can be the same or different from the second assisting device. The assisting devices include overtube, single balloon overtube, double balloon overtube, spiral overtube, motorized spiral overtube, G-EYE endoscope system, and NaviAid balloon system. The first and the second assisting device can be NaviAid balloon system in one embodiment. The first assisting device is one of overtubes and the second assisting device is NaviAid balloon system in another embodiment. The pairs of the first and the second assisting devices are single balloon overtube and double balloon overtube, two overtubes, two double balloon systems, and two single balloon systems, one single balloon system and one double balloon system, two G-EYE endoscope systems, two NaviAid Balloon systems, and One G-EYE endoscope system and one NaviAid Balloon system.

In one embodiment, the diseases for this treatment include one of diabetes, obesity, nonalcoholic fatty liver disease, digestive diseases, cancers, tumors. The tissues applicable to such a treatment include nonvascular lumens, digestive lumens, duodenum, jejunum, ileum, colon, cancers, tumors.

It is understood that both the foregoing general description and the following detailed description are exemplary and explanatory only and are not restrictive of the present invention as claimed.

### BRIEF DESCRIPTION OF THE DRAWINGS

FIG. 1 illustrates various abdominal organ and various anastomosis sites. The jejunum and ileum, the duodenum and ileum, the duodenum and jejunum, the stomach and colon, the stomach and small intestines, jejunum and cecum, duodenum and cecum, ileum and cecum, and the small intestines and colon are potential anastomosis sites, in accordance with various embodiments.

FIGS. 2A-2C illustrate side and vertical views of MAD components, in accordance with various embodiments. The disc has diameter d and height h, and the ring has outside diameter D, height H and hole diameter DH.

FIGS. 3A-3C illustrate side and vertical views of assembled MAD with outside diameter OD, inside diameter DH, and height H with polymeric heat shrink tube and guiding elements, in accordance with various embodiments. The knots of guiding elements are located at the middle of the side wall of the heat shrink tube, in accordance with various embodiments.

FIGS. 3D-3F illustrate an assembled MAD with polymeric heat shrink tube and guiding elements. The guiding elements are located close to the joint of the MAD in the heat shrink tube, with the knots of guiding elements located at the top surface of the heat shrink tube and the lines exiting from the bottom surface of the heat shrink tube (e.g., thru the thickness), in accordance with various embodiments.

FIGS. 3G-3H illustrate another MAD assembly of FIG. 3B but the lines are exiting from the middle side wall of the heat shrink tube (e.g., thru the half thickness).

FIGS. 3I-3J illustrate an assembled MAD with a single guiding line that has one knot to attach to one end and the line goes through the holes on the other end to connect both ends, in accordance with various embodiments.

FIGS. 3K-3L illustrate an assembled MAD with a looping method of a single guiding line to connect both ends free of using knots, in accordance with various embodiments.

FIGS. 3M-3N illustrate an assembled MAD, in which a single line loops through two holes located at each very open end of the joint of the MAD in the heat shrink tube, in accordance with various embodiments.

FIGS. 3O-3P illustrate an assembled MAD, in which two separated guiding lines loop through (in and out) two holes at each end to connect the MAD ring, in accordance with various embodiments.

FIGS. 4A-4B illustrate an end piece design for guiding line attachment and joint parts of MAD ring assembly, in accordance with various embodiments. The eye loop of hook ring can be placed in the middle height or at top or bottom of the magnet, in accordance with various embodiments.

FIG. 4C illustrates an end piece in detail, in accordance with various embodiments. The hook ring can be directly attached to the magnet or attached to a thin disc first, then the thin disc with hook ring attached to the magnet, in accordance with various embodiments.

FIG. 4D-4E illustrate an end piece design, in accordance with various embodiments. The magnet has two shallow grooves parallel at each side. In this example, the grooves are located in the middle of the height. The hook ring can snap into the groove to be fixed there, and the eyelet loop can be located inside or partially inside of groove, in accordance with various embodiments.

FIG. 4F-4G illustrate an end piece design, in accordance with various embodiments. With the same mid-height grooves, an arc shape line is attached to the grooves to leave a gap for guiding element to thread through, in accordance with various embodiments.

FIG. 5A illustrates a magnetic disc with a chamfer located at four corners symmetrically or asymmetrically (two on each flat surface), in accordance with various embodiments.

FIG. 5B illustrates a side view of a rounded disc without other geometric features, in accordance with various embodiments.

FIG. 5C is a side view of a chamfered disc with specific dimensions of A, B, C, and D, in accordance with various embodiments.

FIG. 5D is a side view of a filleted disc with specific dimensions of A, B, C, and D, in accordance with various embodiments.

FIG. 5E is the side view of a trapezoidal or beveled disc with specific dimensions of A, B, and C, in accordance with various embodiments.

FIG. 5F is a top view of an oblong disc with specific dimensions of A, B, and C, in accordance with various embodiments.

FIG. 6A illustrates a MAD delivery system with an assembled MAD in deployable linear state in delivery sheath, in accordance with various embodiments. There is a single guiding line at each end of magnets assembly, in accordance with various embodiments.

FIG. 6B illustrates a MAD delivery system with an assembled MAD in deployable linear state in delivery sheath, in accordance with various embodiments. There are folded (double) guiding lines at each end of magnets assembly through the eye loop, in accordance with various embodiments.

FIG. 6C illustrates a MAD delivery system with an assembled MAD in deployable linear state partially covered by delivery sheath to hold MAD assembly and pushing tube together, in accordance with various embodiments.

FIG. 7A-7B illustrate a MAD delivery system with an assembled MAD in a deployable state in a catheter, endoscopic overtube, endoscope or laparoscope, in accordance with various embodiments.

FIG. 8A illustrates first and second MAD delivery systems with an assembled MAD in deployed state in body lumens with guiding elements still attached to MAD, in accordance with various embodiments.

FIG. 8B illustrates a MAD delivery system with an assembled MAD in deployed and reshaped (final) state in the body lumen with guiding elements still attached to MAD, in accordance with various embodiments.

FIG. 8C illustrates first and second MAD delivery systems with assembled MADs in deployed and final state in the mating stage with guiding elements still attached to MADs, in accordance with various embodiments.

FIG. 8D illustrates first and second MAD delivery systems with assembled MADs in deployed and mated stage, and being ready for detaching the MADs, in accordance with various embodiments.

FIG. 8E illustrates the compression of two body lumens by two MADs without guiding elements attached to MAD, in accordance with various embodiments. The MAD delivery systems are withdrawn, in accordance with various embodiments.

FIG. 8F illustrates necrosis, formation of the anastomosis, and MADs release from anastomosis site, in accordance with various embodiments.

FIG. 9 illustrates a MAD having discs axially (thickness direction) magnetized with north poles or south poles in alternating vertical directions, in accordance with various embodiments. MAD components at the open ends of MAD are opposite poles attracted to each other. The number n is an even number, in accordance with various embodiments.

FIG. 10A illustrates a MAD pair in vertical direction of discs axially magnetized with north poles in upper side, in accordance with various embodiments. Two MADs are attracted to each other with opposite poles between contacting surfaces, in accordance with various embodiments.

FIG. 10B illustrates a MAD pair having discs axially magnetized with north and south poles alternately arranged in the ring, in accordance with various embodiments. Two MADs are attracted to each other with opposite poles. The letters S and N represent the opposite poles at contacting faces. The guiding lines are attached through the eye loops, in accordance with various embodiments.

FIG. 10C illustrates an embodiment of a MAD pair having discs axially magnetized with north and south poles alternately arranged in the ring. Two MADs are attracted to each other with opposite poles. The letters S and N represent the opposite poles at contacting faces. The removable guiding lines are attached through the heat shrink sheath with knots, in accordance with various embodiments.

FIG. 10D illustrates an embodiment of a MAD pair having discs axially magnetized with north and south poles alternately arranged in the ring.

The magnets include chamfers and the chamfers align along the ring direction, in accordance with various embodiments.

FIG. 10E illustrates an embodiment of a MAD pair having chamfered discs axially magnetized with north and south poles alternately arranged in the ring. The removable guiding lines are attached to the sheath with knots on the top surface and exit the sheath at the middle of side wall, in accordance with various embodiments.

FIG. 10F illustrates an embodiment of a MAD pair having oblong discs axially magnetized with north and south poles alternately arranged in the ring. The removable guiding lines are attached to the sheath with knots on the top surface and exit the sheath at the middle of side wall, in accordance with various embodiments.

FIG. 11A illustrates an inside view of an anastomosis formed after MADS implantation, in accordance with various embodiments.

FIG. 11B illustrates an external view of the intestinal anastomosis at necropsy, in accordance with various embodiments.

FIG. 11C illustrates an inside view of an anastomosis joint line, in accordance with various embodiments.

### DETAILED DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

Embodiments of the present invention relate to a magnetic anastomosis device (MAD), a MAD delivery catheter, methods of delivery of the catheter in targeted body lumen, and methods of treatment for diabetes, obesity, digestive diseases, cancers, and tumors, with the MAD delivery catheter. The delivery catheter includes a combination of a magnetic anastomosis devices and their delivery catheters. The deployable magnetic devices are used for creating anastomoses between two body lumens or organs (e.g., between two body lumens, between two organs, or between a body lumen and an organ) to be joined for the purpose of redirecting bodily fluids. The term “between two body lumens” can include between two different body lumens or between two segments of a single body lumen (e.g., between proximal and distal intestine). An anastomosis can treat disease in any suitable way, such as by bypassing an obstruction caused by a tumor, creating a new pathway for foods, urine, blood, bile, or pancreatic enzymes, or such as by bypassing or partially bypassing a segment of the gastrointestinal tract in order to decrease nutrient absorption, alter intestinal microbial flora, affect hormonal changes (e.g., incretin), or a combination thereof. The body lumens and organs includes small and large intestines, cecum, stomach, biliary duct, esophagus, colon, duodenum, jejunum, ileum. The methods involve delivery of the magnetic devices to targeted body lumen in the human body by endoscopic and/or laparoscopic techniques using an access device such as an endoscope, overtube, colonoscope, enteroscope, laparoscope, overtubes, single balloon overtubes, spiral overtubes, double balloon endscope system, G-EYE endoscope system and their combinations.

Embodiments of the present invention are directed to the treatment of diseases by delivery of a MAD through access devices with MAD delivery systems. Two separately delivered MADs are placed at two pre-selected locations of the treatment and coupled together to create anastomoses.

The MADs are used for creating anastomoses between body lumens and/or organs. The methods involve delivery of the magnetic devices such as magnetic rings to targeted lumens or organs in the human body by endoscopic and/or laparoscopic techniques. The MAD can be delivered in a low profile via linear configuration using catheter, endoscope, colonoscope, enteroscope, laparoscope, overtubes, single balloon overtubes, spiral overtubes, double balloon endscope system, G-EYE endoscope system and their combinations. Upon delivery, the MAD assembles into the ring shape in the body lumen using guiding elements, and can be paired to a second MAD ring to join tissues, such as tissues of the gastrointestinal tracts. Optionally, the first and second MAD can be delivered simultaneously, or at different times. The blood supply to tissues in-between two MAD rings is cut off by compression pressure. The tissues can be allowed to necrose and the surrounding tissue to heal together over time and form an anastomosis.

The removable guiding elements attach to the periphery of the magnet assembly, to the inside of the polymer heating shrink tube (PHST), and to the surface of the PHST through the tube. The guiding elements are selected from single or multiple polymer filaments, sutures, and metal wires. The guiding element materials can be selected from a polyester, a polyamide, a polyethylene (e.g., UHMWPE), polypropylene, and a combination thereof. The number of the guiding elements can be any suitable number, such as from 1 to 10, or 2 to 4. The guiding element diameter can be less than 0.5 mm.

Various embodiments of the present invention simplify the medical procedure of traditional gastrointestinal bypass by avoiding open surgery. In one embodiment, this procedure is simply to deliver the first MAD into the first location of small intestine such as jejunum by typical endoscopy technique through mouth and stomach; and to deliver the second MAD into the second location of the small intestine like ileum to be joined with typical endoscopy technique through anus and rectum. Guiding elements of the device can reshape the delivered linear MAD into a ring configuration, then bring two MAD rings (devices) together. The blood supply to the tissues in-between two MAD rings is cut off by compression pressure. The tissues can be allowed to necrose and the surrounding tissue to heal together over time and form an anastomosis.

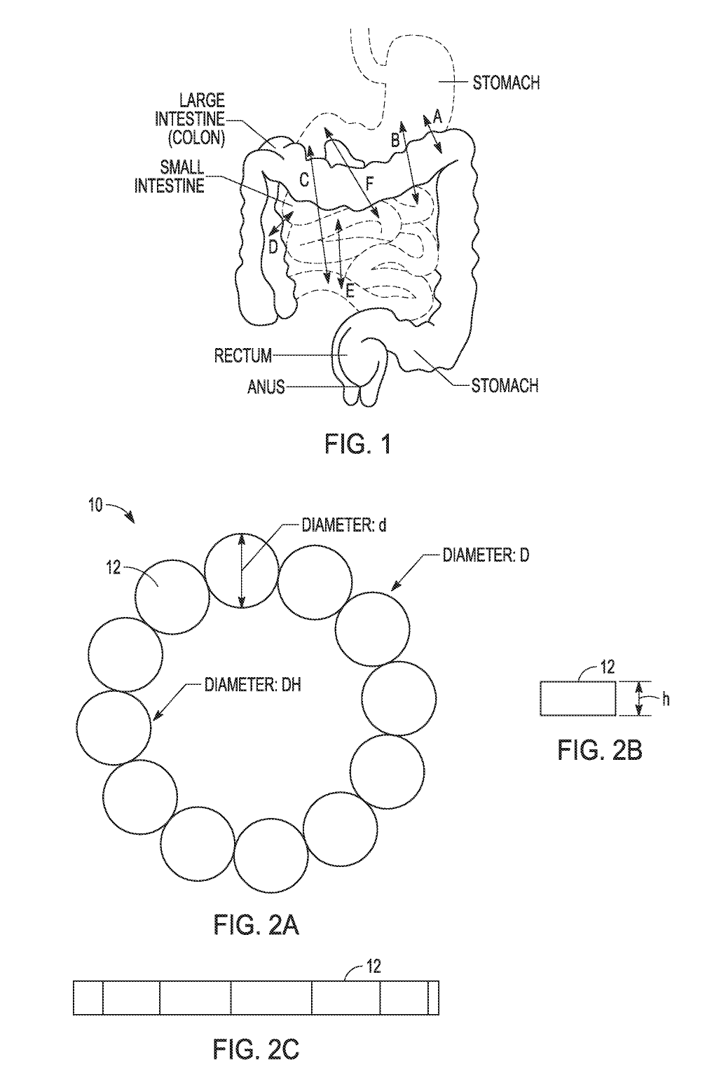

The MAD device specification is largely dependent upon the intended use for creation of anastomosis. There can be several key design parameters to consider for a given anatomic target site with known tissue thickness: strength (force) of magnets, sizes of MAD such as outside diameter and inside diameter, magnet shape and size, device delivery profile, and so on. After the specification is determined, the MAD device design can be developed to treat or to create anastomosis between tissues as illustrated in FIG. 1 for examples. The double-sided arrows in FIG. 1 illustrate some options for anastomosis sites. Examples include the jejunum and ileum (arrow e), the duodenum and ileum (arrow c), the duodenum and jejunum (arrow f), the stomach and colon (arrow a), jejunum and cecum, duodenum and cecum, ileum and cecum, proximal and distal jejunum, proximal and distal ileum, the stomach and small intestines (arrow b), and the small intestines and colon (arrow d) are potential anastomosis sites.

The MAD device can have any suitable form. For example, FIG. 2 illustrates an embodiment including a magnet assembly in a ring form. In this example, the MAD 10 was formed by the assembly of circular disc magnets 12 with the disc diameter d and thickness h and open ends. The formed MAD had outside diameter D and device hole diameter DH. The diameter of resulting anastomosis is approximately equal to the device diameter D. The overall magnetic force of the MAD device can be proportional to the number of discs used, dimensions of the magnets, and the mass of the magnets. Generally the more mass, the stronger the magnet is. In some embodiments, the MAD forces between two MAD rings with a gap of 1 mm are about one pound force from two 24-disc rings of 2.75 mm×2 mm (diameter×thickness) discs, and about two pounds force from two 18-disc rings of 3.75 mm×2.75 mm×2 mm (length x diameter x thickness, an oblong shape disc). The forces were measured between a fixed MAD ring and a floating MAD ring supported by a nonferrous flat surface at a distance of 1 mm. The two rings were in parallel and the floating ring would self-align with the fixed ring in N-S pair at vertical direction.

Due to a desire for a low delivery profile that is compatible with a particular access device such as an endoscope work channel, a balance between the d (of disc diameter) and h (of disc thickness) of the magnet has to be selected, and optionally a rare earth magnet can be selected. Within the profile limit, the largest magnetic diameter possible is preferable since it provides more contact surface area between the rings when disc is round. The ratio of d/h (diameter/thickness or width/thickness if not round like oblongs) can be about 0.5 to about 3, or preferably, about 1.10 to about 1.75. As to the physical shape of the magnetic disc itself used in making the MAD, they may be round or oval or oblong, when viewed from the top or bottom of the ring and it can be desirable to have certain amount of corner radius (e.g., arc) at the edge between circular surface and side wall of the disc. The radius may facilitate passage through a smaller diameter working channel of an access device and may result in a compression pressure gradient on tissues and may help healing by preventing abrupt pressure change during anastomosis formation. The magnets used in fabricating the MAD can be magnetized either axially or diametrically if the magnet is in disc shape. The outside diameter D can be any suitable diameter, such as about 1 mm to about 50 mm, or about 5 mm to about 30 mm. Which diameter of MAD device to be used in the treatment can depend on patient size and patient situation and needs. Typically, smaller diameter may bypass less body fluids through the anastomosis, which, for a weight loss treatment, may mean less weight loss after the procedure.

Rare earth magnets such as neodymium magnets are the most powerful permanent magnets. They are composed mainly of neodymium (Nd), Iron (Fe) and Boron (B), and referred to as NdFeB magnets. The magnetic strength is indicated by N rating that refers to the maximum energy production of the material that the magnet is made from. The grade of neodymium magnets is generally measured in units millions of Gauss Oersted (MGOe). A magnet of grade N42 has a maximum energy production of 42 MGOe. The higher the grade, the stronger the magnet. Suitable grades for the MAD is from N38 to N52, preferably, N42 to N52.

Because neodymium magnets are strong magnets (e.g., high attraction force) and are made of hard, brittle material, they are easily damaged such as from chipping, cracking, or shattering, requiring careful handling during manufacturing and application. Due to the nature of rare earth elements, they are highly reactive under conditions of high temperature and in the presence of water or humidity. The reaction with water can be significantly suppressed by adding sufficient quantities of more noble elements such as cobalt (e.g., to form noble element-modified neodymium), making the speed of the reaction negligible. The magnets in the embodiments of the MAD system are protected with one or more layers of metals like zinc, nickel, copper, gold, silver, nickel-copper-nickel (e.g., a layer of nickel, coated with a layer of copper, coated with another layer of nickel), or any combination thereof; and with polymers like epoxy resins or parylene. The coating can prevent the directly contact of the magnetic material with water, body fluid, or tissue. The coating thickness of Zn or Ni can be in the range of about 3 μm to 15 μm; of Ni-Cu-Ni about 10 μm to about 25 μm total; and of parylene about 5 μm to about 15 μm. The reactivity of the magnet materials can also be shielded by a plating of metal materials such as zinc, nickel, and copper, by coating with polymers such as parylene and PTFE, or by a combination of plating with metals and coating with polymers. Biocompatibility of the magnet can be increased by using, as a top layer of the MAD magnets, gold or silver plating, a polymer coating such as parylene, epoxy or PTFE, or a combination of metal plating and polymer coating, with preferred coating thickness of gold or silver about 0.1 μm to about 15 μm, of epoxy about 10 μm to about 30 μ, of parylene about 5 μm to about 15 μm, and of PTFE about 0.25 μm to about 1.0 mm. Plating and coating on the magnetics can be dual-layer or higher-layer coatings, in which the layers are coated separately, i.e., one after another. For a dual-layer coated disc, for examples, the magnet disc can be coated with Zn first, then coated with gold; or, first coated with nickel, then coated with parylene. Examples of higher-layer coatings are coating nickel, coating with copper, and then coating with nickel (e.g., Ni-Cu-Ni); or coating with nickel, then coating with gold, and finally coating with parylene that is top surface material; or to coat Zn first, then to coat gold, and finally to coat parylene. Additional layers may be added; for example in one embodiment each magnet disc is individually coated in the following order: nickel, copper, nickel, gold, and parylene. In various embodiments of the MAD system, the magnets are further protected by a layer of durable plastic heat shrink sheath to hold the string of discs together and to protect from damage to the top surface layers of the magnets.

In some embodiments, during heat shrink tube assembly, the magnets are exposed to heat when the MAD is inside the plastic shrink sheath during the shrinking and wrapping process. However, neodymium magnets are sensitive to temperature exposure and can become demagnetized at high temperature. Neodymium magnets can have 80° C. listed as the actual maximum operating temperature. Therefore, in some embodiments, a maximum operating temperature for the magnets used in the MAD is >80° C., ≤100° C., or ≤110° C. The suitable high temperature grades are N42SH to N52SH, for example, N48SH.

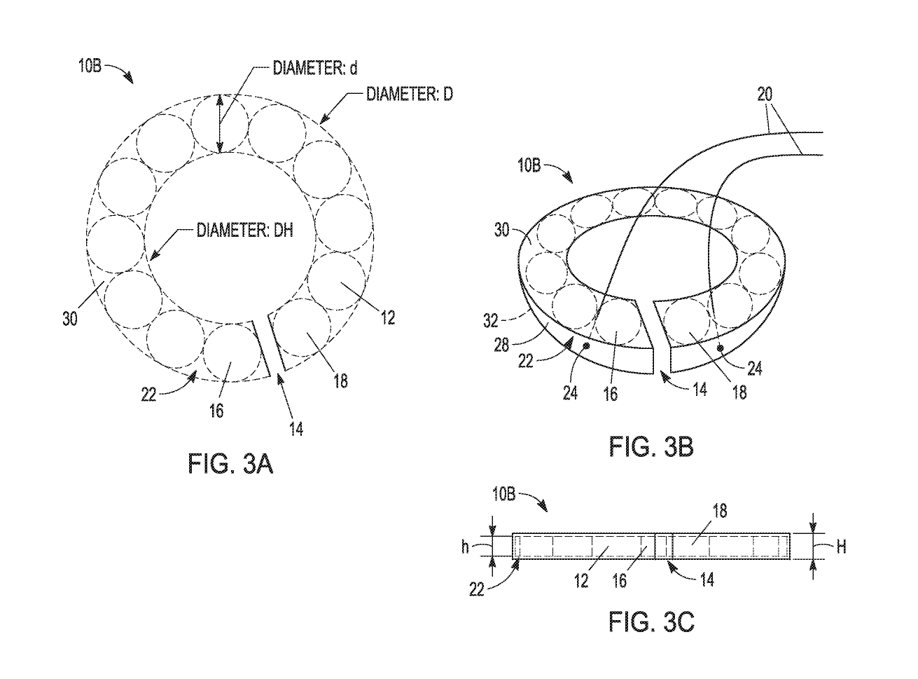

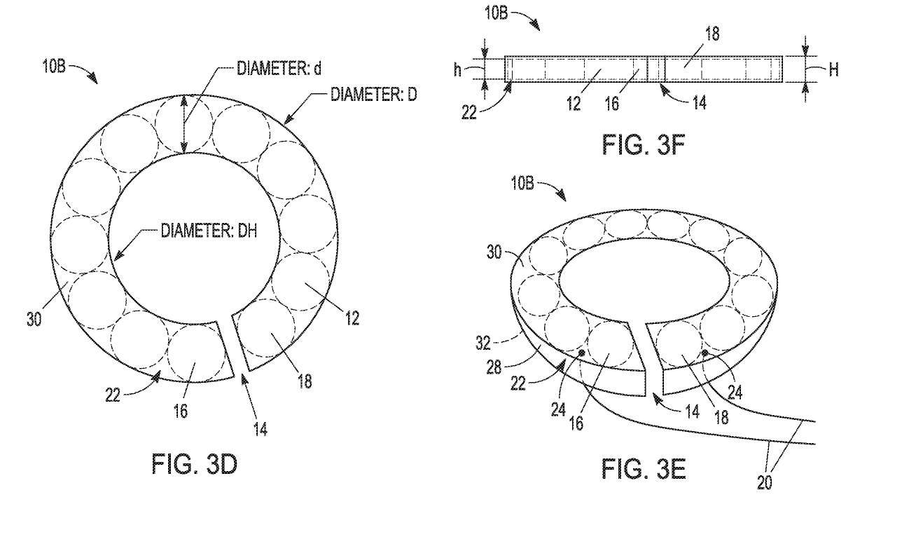

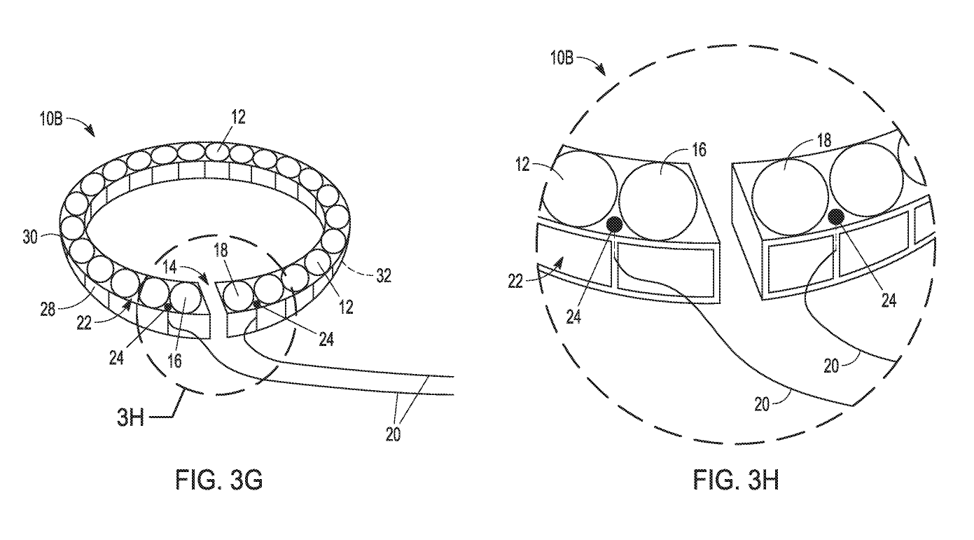

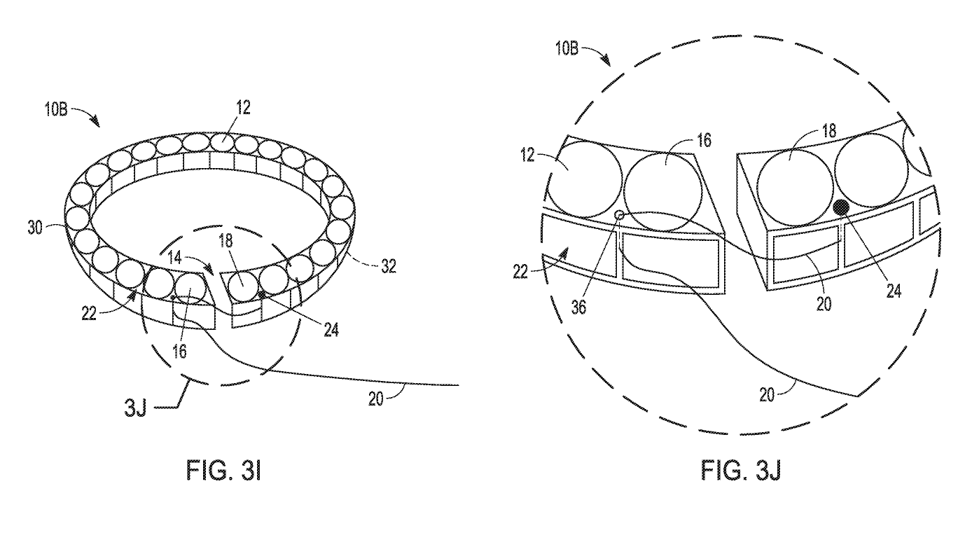

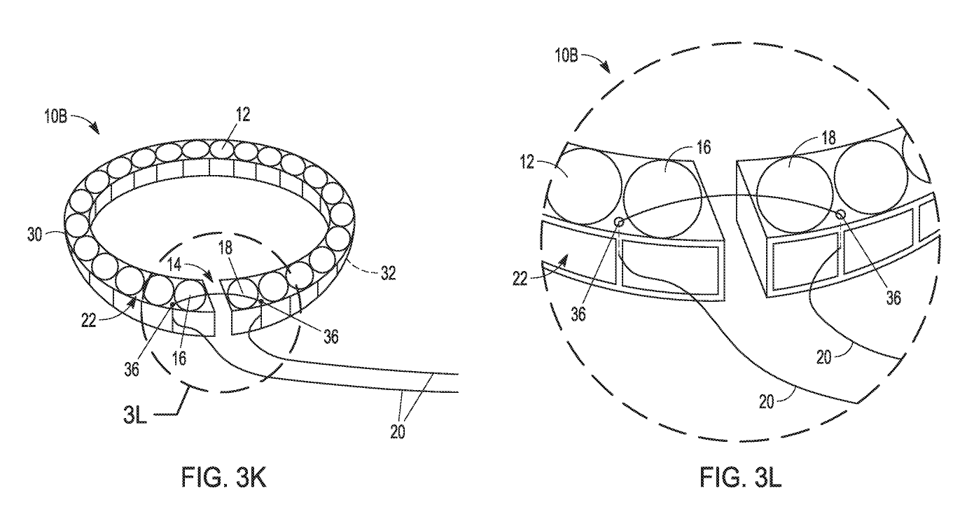

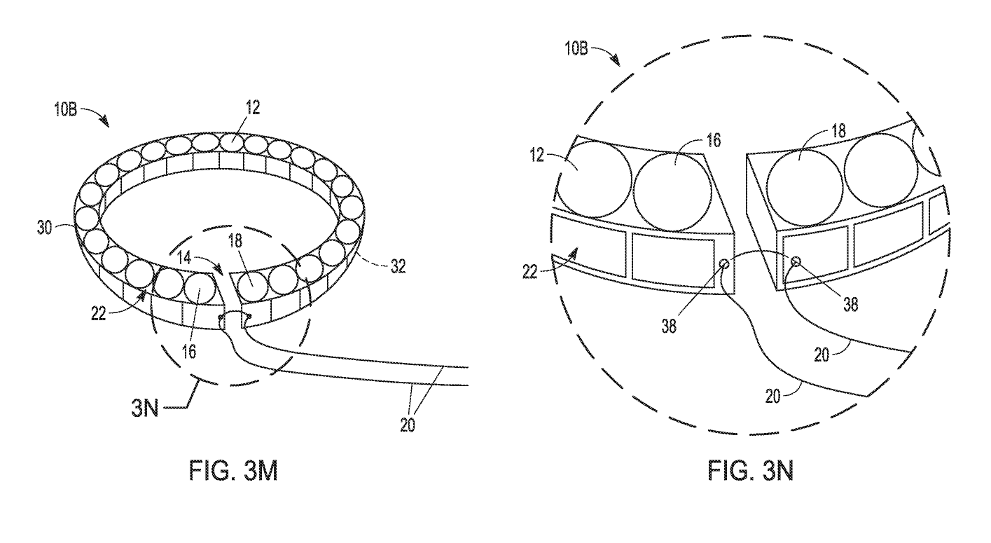

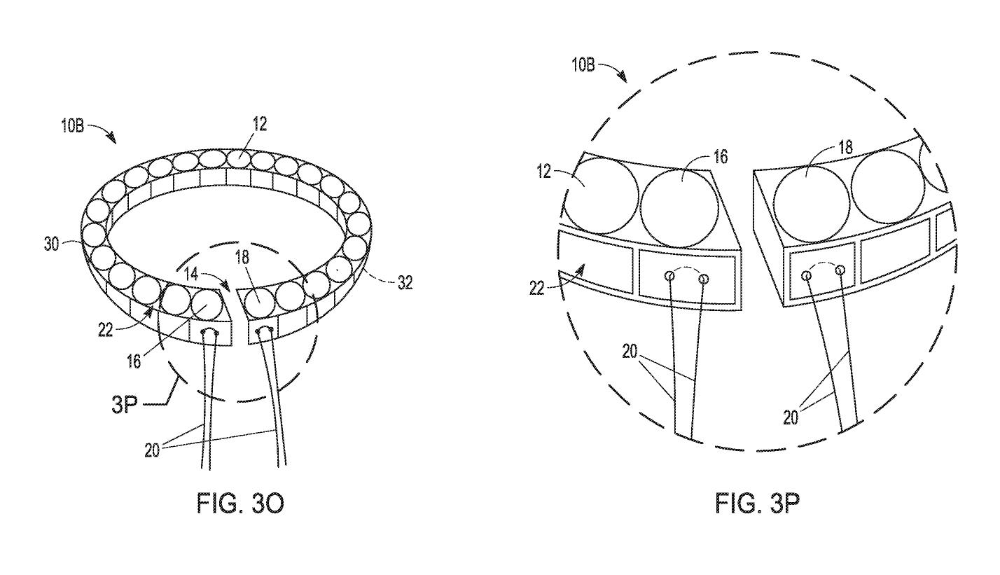

An embodiment of a more detailed assembly is illustrated in FIGS. 3A-P, in which a heat shrink tube was used to fix the magnets into place as an enclosure and guiding elements. The MAD 10B in FIGS. 3A-P is in the deployed and final shape state. There is an opening 14 in assembled MAD ring for transforming to linear configuration before delivering through scope. The opening is joined/closed by two end discs 16 and 18 with opposite magnetic polarity (e.g., N-S). The guiding elements 20 are located close to the joint of the MAD in the heat shrink tube 22 with the knots 24 or loops. After delivering into a body lumen, the MAD can be re-circularized via guiding elements and detached from the guiding elements for releasing the rings. Because of the need of recircularization, a round end is preferred for the contact surfaces of the discs in the MAD ring, in which the round contact surfaces between the discs act as a hinge point for a smooth movement in the transformation of circle ring to linear form and back to circle ring. Theoretically, half circle round end will give 180 degrees moving angle.

The size ratio between the circumference of the heat shrink tube inside diameter and the perimeter of the magnet C_{ID}/P_{cs} can be 1 to 2. The heat shrink tube can be flexible, conformable, and durable, with a wall thickness of 0.05 mm to 0.75 mm, or 0.15 mm to 0.50 mm. The selected heat shrink tube can be conformable to the shape of the magnets and wrapped tightly onto the magnets after heat shrinking. With alternating magnetic pole arrangement and polymer heat shrink sheath the resulting MAD ring has identical top and bottom surface that provides non-preferential mating surface. The selected heat shrink tube can be durable to protect magnets from any possible damages due to any brittleness of the magnets. The selected heat shrink tube can be strong enough (e.g., have sufficient tear resistance) to hold the guiding elements (e.g., which can be attached via knots) and to not tear during manipulation of the magnets from straight to a ring shape. The selected heat shrink tube can retain or at least partially retain the MAD ring shape during delivery procedure to assist the transformation from linear form back to ring shape.

The diameters D and DH include the heat shrink tubing. Part of the guiding elements are shown in the figures, shown as a line (e.g., wire) attached to the heat shrink tube at or close to the first and the last open magnet position. The line may be metal wire such as stainless steel wire or nitinol wire, or polymeric mono- or multi-filaments like braided fibers. In considerations of line dimensions versus their strength and delivery profile, the line diameter can be 0.05 mm to 1.0 mm, 0.10 mm to 0.50 mm. The guiding elements can attach to the periphery of the magnets and inside of the polymer heating shrink tube (PHST) or to the PHST near the end magnets. The guiding elements can attach to the periphery of the magnets between the end magnet and the second magnet to the end magnet and through the PHST tube (e.g., with knots), with the point of connection being at the side walls 28 (FIGS. 3A-C), at the top surface 30 (FIGS. 3D-F), or a combination thereof. In these two particular examples, the guiding element lines exit from the top surface of the PHST in FIGS. 3A-C and from the bottom surface 32 of the PHST (thru the thickness) in FIGS. 3D-F. The guiding elements can be selected from single or multiple polymer filaments, sutures, and metal wires. The guiding elements can be selected from a polyester, a polyamide, a polyethylene (e.g., UHMWPE), a polypropylene, and a combination thereof. The number of the guiding elements can be from 1 to 10, or 1 to 4. The guiding elements enable the MAD configuration transformation from linear (e.g., the delivery configuration), to ring (e.g., the final stage configuration). They also facilitate manipulation of the MAD alignment during the process of mating the formed rings. The guiding elements are removable from the delivery catheter.

In another embodiment, the guiding elements can attach to the periphery of the magnets between the end magnet and the second magnet to the end magnet and through the PHST tube (e.g., with knots), with the point of connection being at the top surface and the lines exiting at the middle of the side wall (FIGS. 3G-H). The guiding element lines are detached via a pull-through knot mechanism.

In some embodiments, the guiding elements can be a single line attachment. FIGS. 3I-J illustrates a top surface knot connecting point 24, and the line exiting from the middle side wall and looping thru a hole 36 at the top surface of the other end and exiting from middle side wall.

FIGS. 3K-L illustrate a looping connection between two ends with the line from the top wall (holes 36) to side wall without any knots. FIGS. 3M-N are another example of the looping connection but at different locations; the line thru the holes 38 that are located at the very open end of heat shrink sheath of the first and last magnets at middle side wall position. The guiding element lines can be detached by pulling away the lines thru the holes or by tearing the heat shrink tube wall.

In another embodiment, the guiding elements can attach to the periphery of the magnets between the end magnet and the second magnet to the end magnet and through the PHST tube (e.g., with close distance holes), with the loop of connection at the middle of the side wall (FIGS. 3O-P). There are 4 lines with 2 loops in this guiding element (4 pulling lines system). The detachment mechanism of the guiding elements is to pull both lines between two holes at each end to tear the PHST wall (between two holes). Generally, the closer the distance between two holes, less force is needed to tear away the wall material.

Different diameter MADs may be assembled with various numbers of the magnets, with various diameter (d) of the magnets, and with various geometric shapes. The number of the magnets can be in the range of 1 to 40, 6 to 30, or 14 to 26. The number of the magnets can be even or odd. With alternating polarities of the magnets, an even number, such as 2, 4, 6, 8, 10, 12, 14, 16, 18, 20, 22, 24, 26, 28, or 30, can be used. With limited work channel diameter of an endoscope, preferred profile of the MAD in linear form is 3.5 mm or smaller. In some exemplary embodiments, the 2.75 mm (Dia.)×2 mm (Thickness) circular disc MAD ring has linear profile about 3.5 mm and 24 mm ring outer diameter with 24 2.75 mm discs, which is compatible with the working channel diameter of 3.7 mm and larger of an endoscope. The MAD ring made of 20 discs of 3.15 mm (Dia.)×2 mm (Thickness) with chamfer features yields about 24 mm ring outer diameter and fits 3.7 mm and larger of an endoscope. An oblong disc MAD ring fits 3.2 mm and larger of an endoscope is made of the oblong discs of 3.75 mm (Length)×2.25 mm (Width)×1.125 mm

(Radius). A MAD ring fits 2.8 mm and larger work channel can be made of the oblong discs of 4.2 mm (Length)×2 mm (Width)×1 mm (Radius). In these exemplary embodiments, the overall length of the oblong discs contributes to the magnetic strength and does not change/has any impact on scope compatibility; the scope compatibility (fitting into work channel) is only determined by the width and thickness with the exception of chamfered discs.

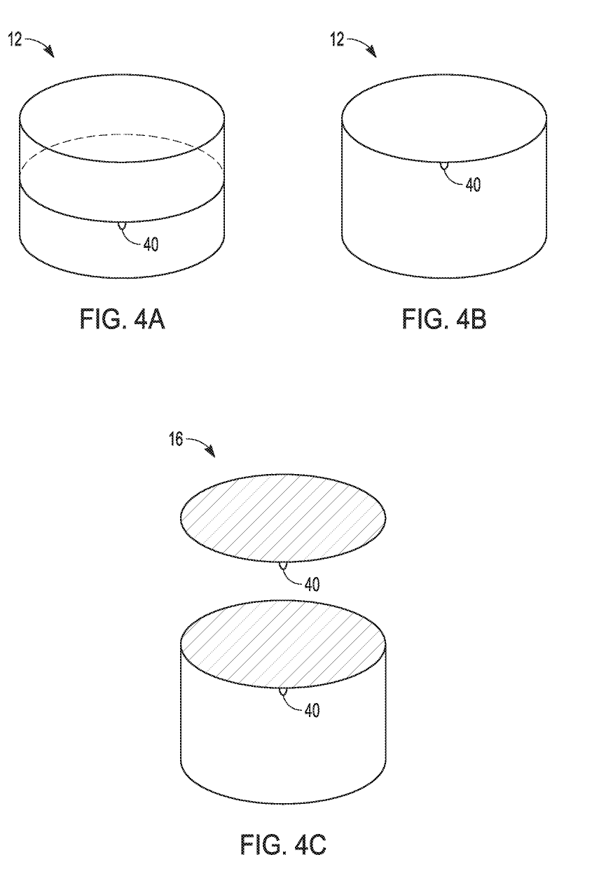

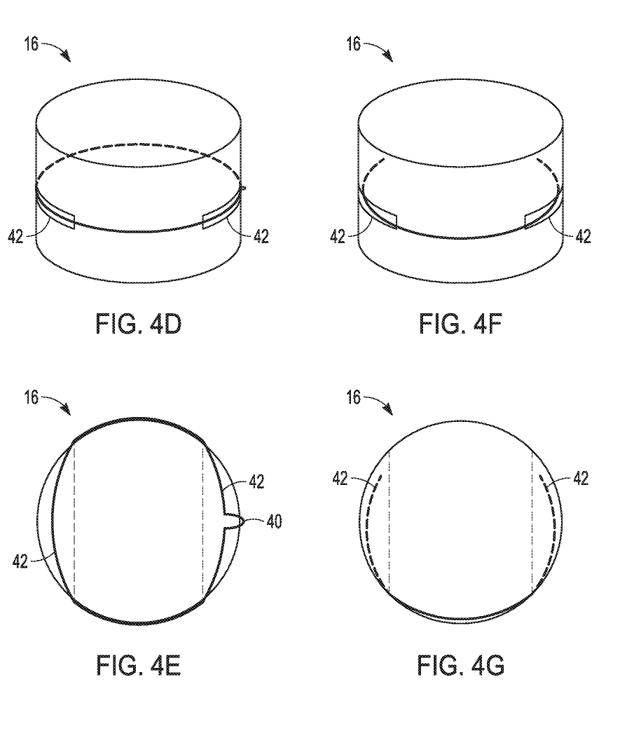

To ensure easy operation for the guiding elements to achieve final MAD configuration and withdrawal of the guiding elements afterwards, various magnets can be specially designed to accommodate the guiding elements, such as the magnets at two open ends of the MAD. As shown in FIGS. 4A-B, a hook ring may be incorporated into the magnetic disc 12 with an eyelet loop 40 to receive the line of the guiding element. The hook ring can be placed in the middle of the disc height (FIG. 4A) or at the top or the bottom of the disc (FIG. 4B). The material of hook ring can include metal wire, plastic filament (e.g., mono- or multiple-filament, braided or not braided), a plastic part (e.g., molded), or a combination thereof

FIGS. 4C to 4G illustrate various embodiments of an end magnet design of a MAD. FIG. 4C show a hook wire fixed onto a thin piece of metal or plastic sheet, which is then attached to a regular magnetic disc. Optionally, the hook ring can be attached directly to the disc without the thin sheet material.

In some embodiments, the magnetic disc can include a feature that holds a hook in place. FIGS. 4D-E illustrate two separated grooves 42 created at the middle height of the disc. A snap ring with proper diameter to the groove width can be attached to the disc at the groove feature areas by snap fitting. Optionally, the hook ring can be permanently fixed onto the magnet by applying adhesive at groove area without increasing profile. The adhesive can include any suitable adhesive, such as a urethane, an epoxy, a UV-curable adhesive, and combinations thereof. With the wire diameter of hook ring less than the groove width, adhesive can be used to hold the wire ends in the inside of grooves. The eyelet loop of the hook ring can be located at the center of one of the grooves, which can be recessed into the groove to reduce overall profile. The hook ring can be located at the middle of the disc height or at any suitable place along the height of the disc. FIGS. 4F-G exhibit another configuration of a hook ring assembly, including an arc-shaped gap between the hook ring and disc wall, so the guiding lines can be threaded through the gap.

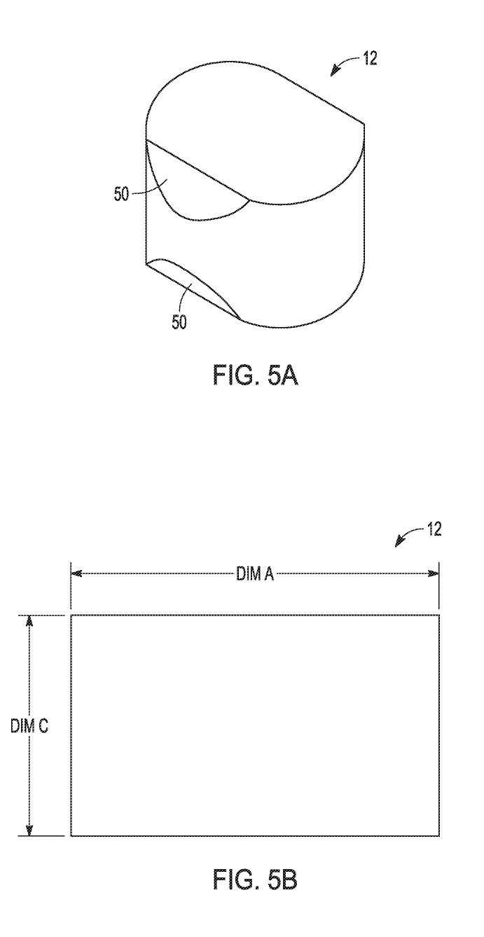

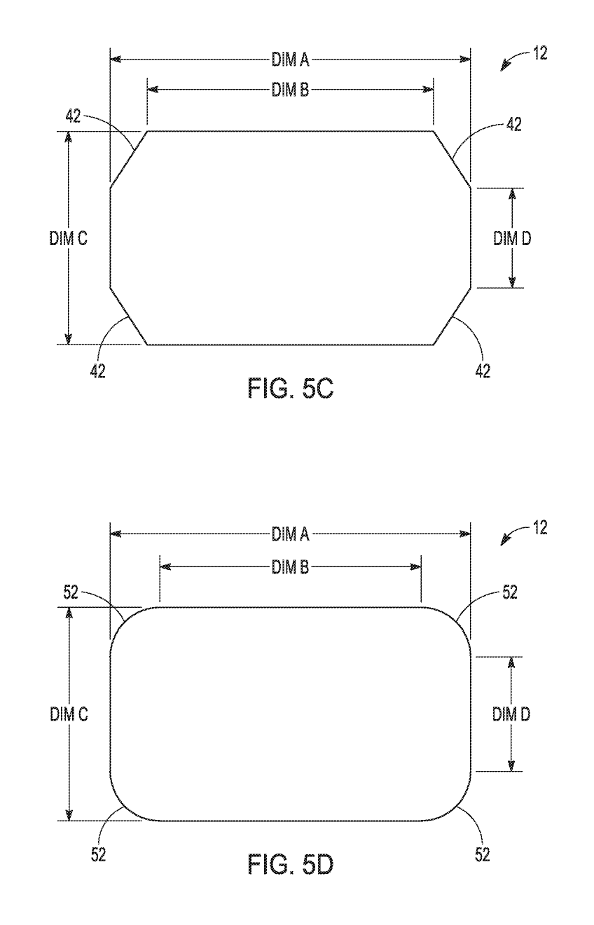

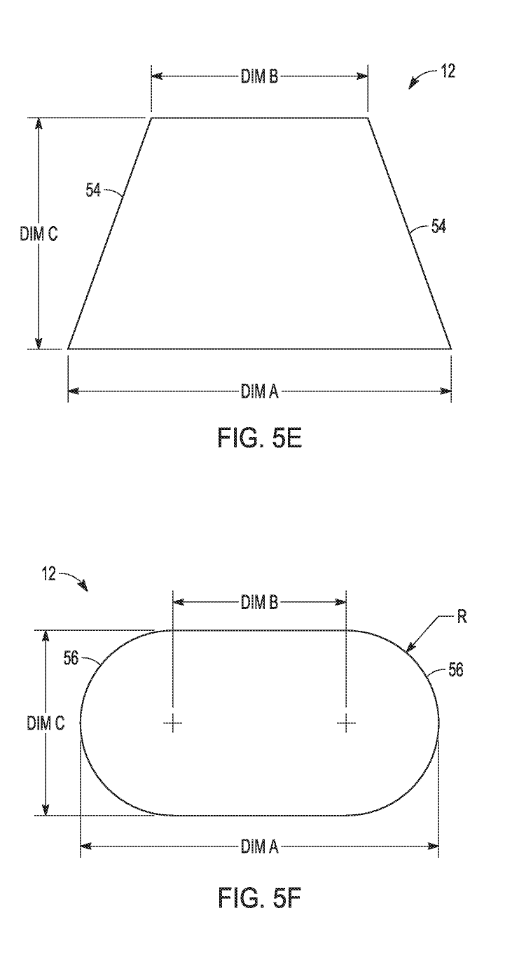

In some embodiments, the magnetic round disc or oblong disc or oval disc or other suitable shape discs can be modified to include a special geometric feature in order to increase or maximize magnetic force and decrease or minimize overall profile, e.g., the entrance profile of a MAD at linear configuration, such as in a scope or catheter. FIG. 5A illustrates chamfer features 50 on the round magnetic disc. The features can be located symmetrically on the disc with two on each flat surface. The side view of the feature is shown in FIG. 5C. The dimensional ratio of Dim B to Dim A is from 0.70 to 0.95 or from 0.75 to 0.90. The ratio of Dim D to Dim C is from 0.30 to 0.60, or from 0.40 to 0.50. With the diameter of 3.15 mm×thickness of 2 mm disc, the linear profile is larger than 3.7 mm; however, with chamfer features on the disc, the linear profile reduces to 3.4 mm that is compatible with 3.7 mm work channel (scope). The previous example refers to a round disc; in other embodiments, the chamfer feature is applied to oval or oblong discs to reduce the profile.

For the purpose of illustrations and comparison, FIG. 5B represents the side view of a regular magnetic disc. FIG. 5C is the side view of chamfered disc. FIG. 5D is the side view of the round disc with fillet features 52, in which the same dimensional ratios of FIG. 5C can be applied. FIG. 5E exhibits the side view of the disc with trapezoid or bevel features 54, in which the ratio of Dim B/Dim A is from 0.2 to 0.6, or preferably, from 0.3 to 0.5.

In one embodiment, the magnetic round disc can be modified to oblong shape, which maintains half circle at each end (FIG. 5F). The rounded ends 56 play a hinge role between the discs in MAD assembly, which enhances the joint mobility of disc to disc and eases the transformation process such as from linear to circle during the procedure. The shape of oblong magnet maximizes its contact surface area or magnetic strength and minimizes its profile for insertion through the endoscope of the MAD ring. The ratio of Dim B/Dim A is from 0 to 0.75, preferably, from 0.3 to 0.6. And the ratio of Dim A/Dim C is from 1 to 2.5, preferably, 1.5 to 2.2. In some exemplary embodiments, 2.8 mm and 3.2 mm work channel compatible MAD rings can be achieved with the designs of Radius 2 mm×thickness 1.6 mm×overall length 4.2 mm disc and Radius 1.125 mm×thickness 1.85 mm×overall length 3.75 mm disc, respectively. In other embodiments, oval or oblong discs are further modified with chamfer or filets to further minimize profile or increase magnet mass while preserving compatibility with smaller access device working channels.

The special geometric features described herein, such as trapezoid or bevel or oblong, can be applied to other shapes of magnets (e.g., other than discs). For example, the special geometric feature can be applied to magnets having a shape of a cylinder, a block, a cube, or a combination thereof. The special geometric feature can be applied to a magnet having a shape that has a profile of a circle, oval, elongated ellipse, oblong, square, rectangle, hexagon, octagon, trapezoid, or a combination thereof. The special geometric feature can reduce the overall profile of the magnet to enable the magnet to fit the working channel of an access device such as an endoscope, overtube, or laparoscopy port.

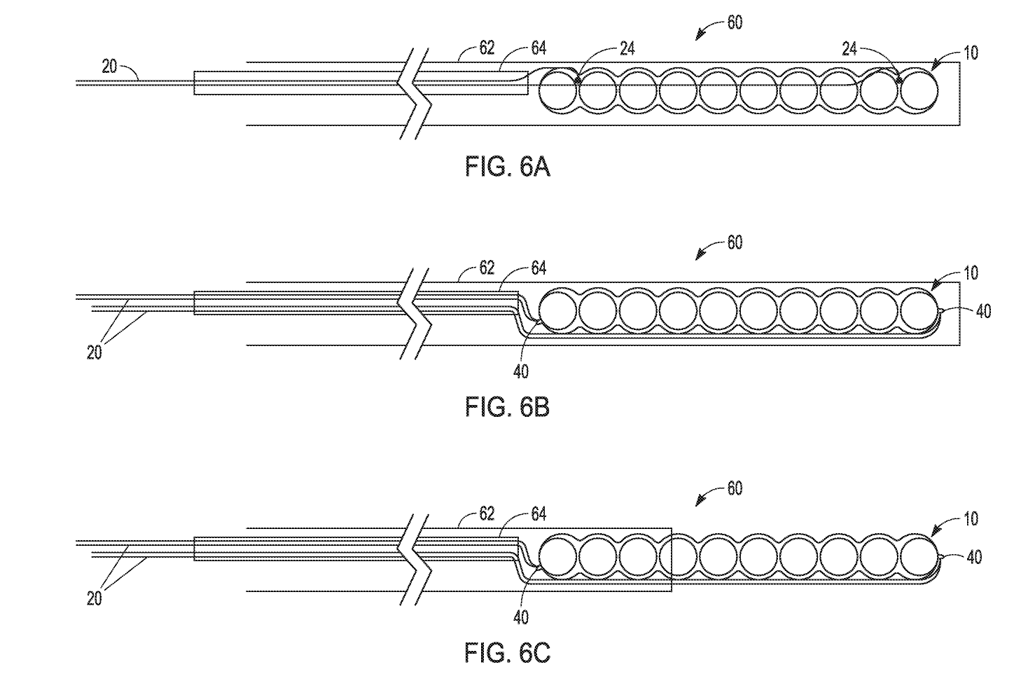

An example of a MAD delivery catheter in deliverable state is shown in FIG. 6A, in which the MAD is in a linear configuration that has the smallest passing profile. The system 60 has a delivery sheath 62 that contains all or some of the components and enables easy delivery via catheter or scope. Inside of the delivery sheath can be included a pushing tube 64, guiding elements 20, and the MAD, wherein the delivery sheath can cover all or part of the assembled magnets. The guiding element can be a single line attached to the end piece, which is removable from the attachment structure. In this embodiment, the two guiding lines are attached to the heat shrink sheath, one close to the last magnets and the other close to the first magnets at each end via a knot. The ratio of the diameter of the knot to the diameter of the hole on the heat shrink sheath is from 1.1 to 2.0, or preferably, from 1.2 to 1.7, with the knot diameter being larger than the hole diameter. The guiding line can be detached from the heat shrink sheath by pulling the line through the sheath to break the connection. Two lines inside the pushing tube control each end of the linear MAD and they run through the pushing tube from one end to another. The pushing tube can have one or more layers of material in overall structure. Typical pushing tube outside diameter is in the range of 3.5 mm to 0.5 mm with an inside diameter of 2.0 mm to 0.3 mm. The pushing tube is longer than a catheter or a scope system. The overall profile of the MAD system should be smaller than the inside diameter of the access device (e.g., endoscope) and the inside diameter of the pushing tube should be able to accommodate the necessary number of lines with low drag/friction on the lines when manipulating the lines. The delivery sheath or introducer can play roles of keeping the distal and proximal wires/lines at designated position and maintaining connection between the MAD and the pushing tube. The delivery sheath or introducer can cover MAD ring partially or fully. The pushing tube pushes the MAD out of the sheath and endoscope and two guiding wires/lines reshape the MAD to ring configuration after the delivery. The pushing tube and guiding lines can then be manipulated to couple two MADs through the walls of the body lumens with the assistance of endoscopic visualization and X-ray images. The line can be detached at the joints of the MAD by pulling the line at the proximal end of the delivery system to break the connection between the sheath and the knot. In some examples, the line is non-stretchable or has very low elongation, like wire (e.g., stainless steel or nitinol wire) and polyethylene fibers such as UHMWPE fibers, such as made from Spectra or Dyneema fiber products. In some embodiments, a single removable guiding element is used that is threaded through the heat shrink tube near the proximal magnet and then attached to the heat shrink tube near the distal magnet using a pull-through knot.

A four line system is shown in FIG. 6B, having a line threaded through the eyelet loop 40 at each open end magnet and the looped line extended to the proximal of the delivery system with two ends. The two pairs of line can be pulled together during the MAD ring formation and can be detached by pulling one of the lines in the pair to remove them completely. The MAD ring can be fully covered by a delivery sheath.

As shown in FIG. 6C, a delivery sheath can be a short sheath like an introducer, which can act as a bridge to connect the magnets and pushing tube in which the introducer covers only a few magnets such as less than five. In this embodiment, the MAD is partially covered by the sheath. The role of the sheath can include maintaining the connection between the pushing tube and MAD, and protecting the position of the guiding lines during the entrance into the access device, such as an endoscope. Once the MAD has fully entered the access device, the sheath can be separated or withdrawn from the MAD ring and the pushing tube can continue to advance the MAD to deploy it to the treatment site.

In one embodiment, the delivery sheath is a short sheath less than 20 inches in length, or preferably, less than 6 inches, covering five or fewer magnetic discs from the proximal end of the linear form of the MAD to the distal end of pushing tube to ensure the connection between the magnets and pushing tube during the entering into the working channel of the access device (e.g., endoscope). Once the ring has fully entered into the working channel, the delivery sheath is separated from the ring by continuing the advancement of the ring with pushing tube.

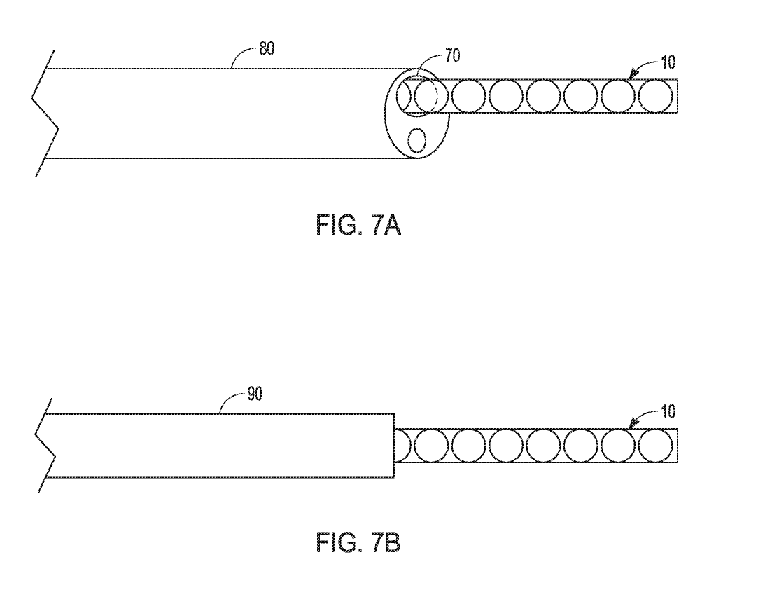

The MAD system in the inside of delivery equipment in FIG. 7 can be delivered into a patient's body lumen with endoscopic procedure through endoscope 80 working channel 70 as illustrated in FIG. 7A. The MAD system can also be delivered through catheter 90, laparoscope port, or endoscopic overtube (in FIG. 7B). It is important to have compatible profile with existing hospital equipment to ensure successful treatment.

In some embodiments, the delivery pushing tube is a dual-layer tube, wherein the outer layer includes an engineering thermoplastic to provide pushability and durability such as unfilled high density polyethylene (HDPE), or filled polyethylene such as barium sulfate-filled HDPE which is radiopaque, or other polymers like polyesters, polyamides and their thermoplastic elastomers; and the inner layer includes PTFE or other fluoropolymers to reduce friction and drag. For example, force transfer efficiency was evaluated by measuring the force at one end of a 9-foot pushing tube, which was coiled into two and half circles with a diameter of 9-inches to mimic the situation in the procedure, and applying pulling force at the other end through a guiding line. The force transfer efficiency was 5-15% with a filled HDPE tube only, and the force transfer efficiency was at 40-50% when the same overall dimension (i.e., ID and OD) dual-layer tube of PTFE inner layer was used. There was about 400% or more improvement for the force transfer from one end to the other end of the pushing tube. The dual-layer tube can be made with co-extrusion process using two different materials, or by inserting a fluoropolymer tube into an outer tube from two separately extruded tubes. The inserted PTFE tube wall thickness can be 0.003″ or higher, preferably, 0.005″ or higher.

In another embodiment, the delivery pushing tube is a single layer tube including a fluoropolymer and having the full dimensions of the pushing tube.

In some embodiments, the delivery pushing tube has an atraumatic tip. The tip material is softer than the pushing tube material or the material of any outer layer therein. The softer tip material can be from the same family of materials as the pushing tube such as a thermoplastic elastomer material or a soft polyurethane material. The softer tip material can have a shore D hardness of less than 55D. In other embodiments, the pushing tube has a stiff tip that can be leveraged against the magnets for manipulation or release of the guiding elements.

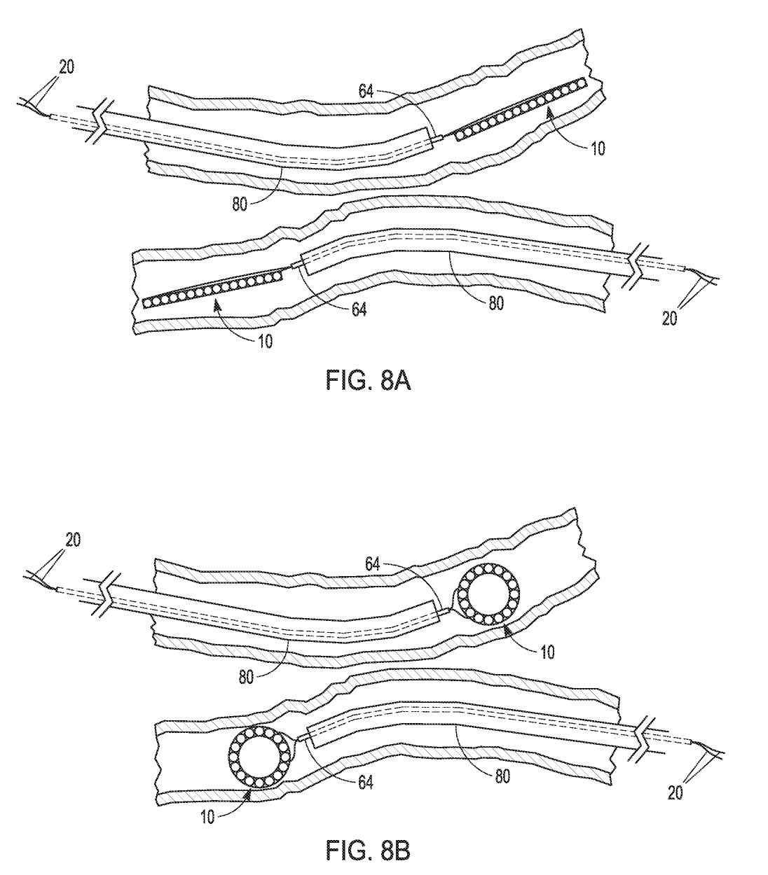

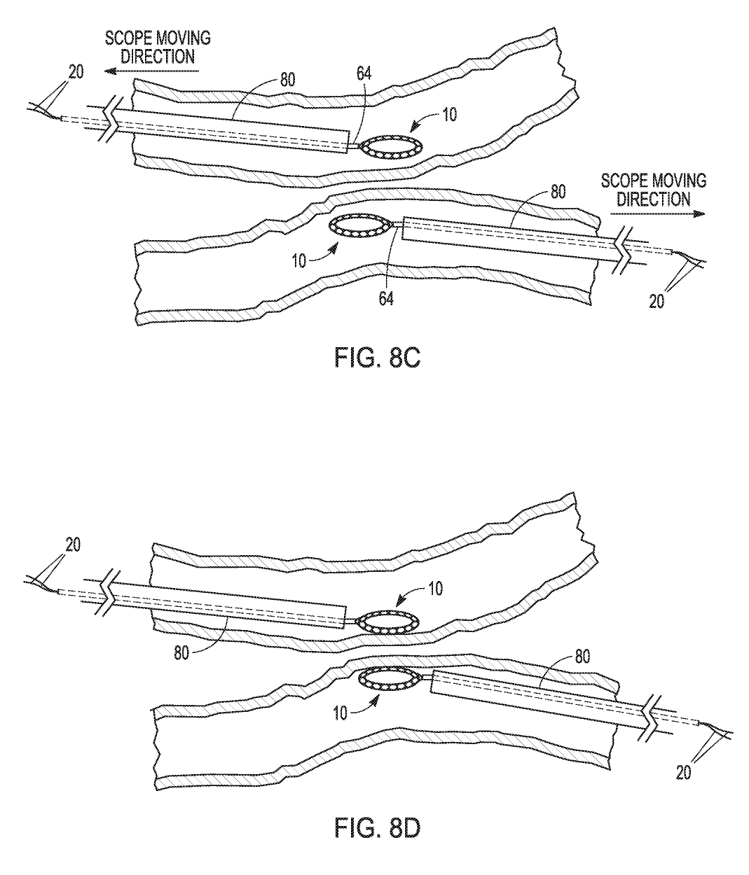

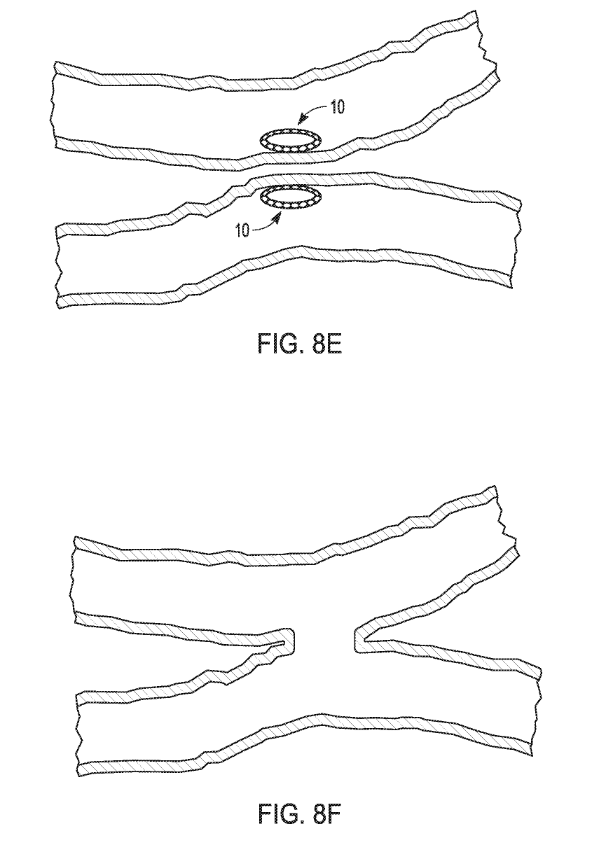

In an embodiment of the anastomosis procedure shown in FIG. 8, using standard techniques and tools known to those skilled in the art of enteroscopy (including for example push and pull techniques, single balloons, double balloons, overtubes, and spiral enteroscopy), the first endoscope was advanced and placed at or beyond the first targeted lumen location; and the second endoscope was advanced and placed at or beyond the second targeted lumen location. The first and the second scopes are proximate to each other, such as less than 2 cm or less than 1 cm apart head-to-head or crossing over distance, which is suitable for MADs to mate successfully. Preferably, this point of proximity can be between the tips of the endoscopes or between points on the shafts of the endoscopes proximal from the tips. In some cases, the tips of the two endoscopes come into close proximity during advancement or manipulation, and the scope light of the other endoscope may even be visible. Then, two scope tips are adjacent to each other, the MADs are delivered from both direction and mated. However, in other cases, it is challenging to bring tips into close proximity during advancement. But as the endoscopes are advanced, the scope shaft loops of the two endoscopes press against each other, and a point on the scope shafts may be identified at which the tips will be in proximity during withdrawal, which is an anatomically convenient site for mating. After the target mating site is confirmed by two orthogonal fluoroscopic views or by rotation through orthogonal views, the MADs are delivered via the working channel of the first and second endoscopes. The figures described in this paragraph are for clear illustration purpose only; actual operation may differ from the specific embodiment described. FIG. 8A demonstrates the exit of the MAD devices from the endoscopes with initial linear configuration near or beyond targeted locations. Ideally both scopes should be positioned closely to each other (e.g., the scopes can be head to head, side by side, or crossing over in relative positions), either at the tips or at an identified target point on the shafts where the tips will be naturally approximate during pull-back, before deploying the MADs as shown in FIG. 8B. With the help of guiding elements, the MAD configuration was transformed to a ring shape as illustrated in FIG. 8B. After finalized MAD configuration, the MAD locations were adjusted to align them face to face with pull-back method (e.g., placing the device ahead of the designated target location initially and then pulling the device back to the target location to meet its counterpart for mating to create the anastomosis) as illustrated in FIG. 8C. The pull-back method can decrease the procedural time required for coupling the MAD pair in the two body lumens, such as by more than half. The pull-back method also reduces the chance of prolapse of MAD ring relatively to the pushing rod 64, avoids the inadvertent trapping of undesired tissue (such as mesentery) in addition to the intended bowel walls. Once done with the alignment, the two MADs were brought together with endoscope maneuver and the magnetic force of the two rings attracted each other to result in a tight contact of the tissues, as shown in FIG. 8D. At this time, the guiding elements are detached from the MADs by either removing the line or cutting the line. The resulting implantation is illustrated in FIG. 8E, and in this case, two small intestine segments were brought together with two MADs. In a week or two after the implantation, as shown in FIG. 8F the anastomosis was formed.

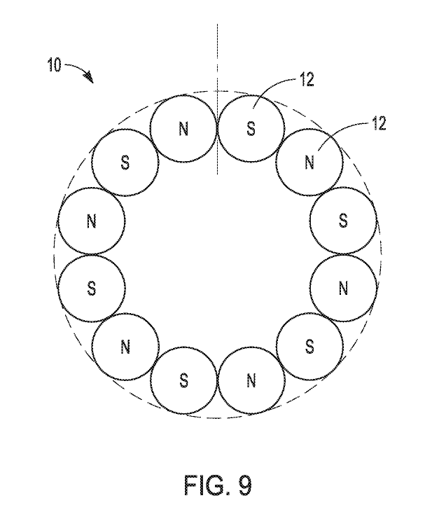

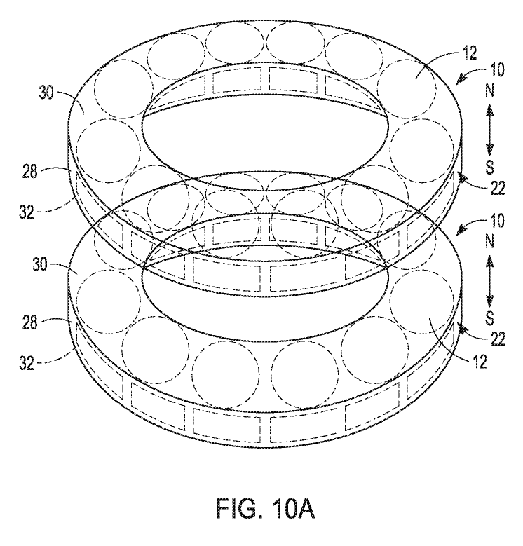

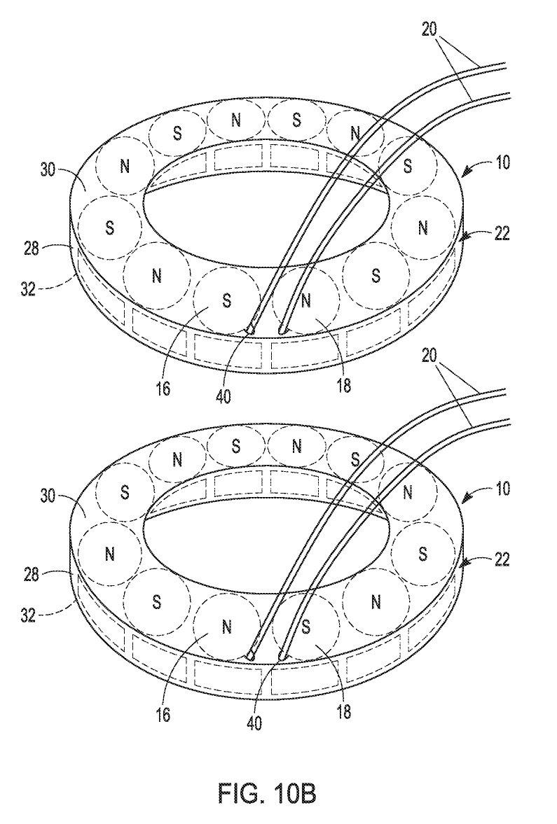

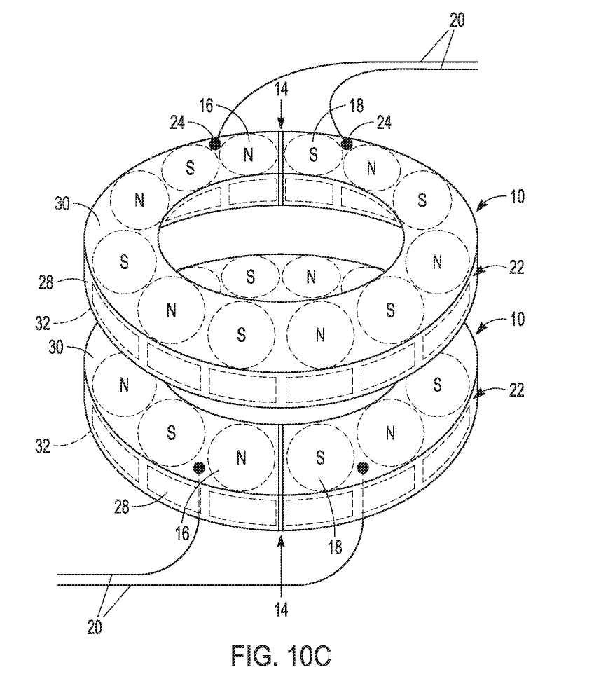

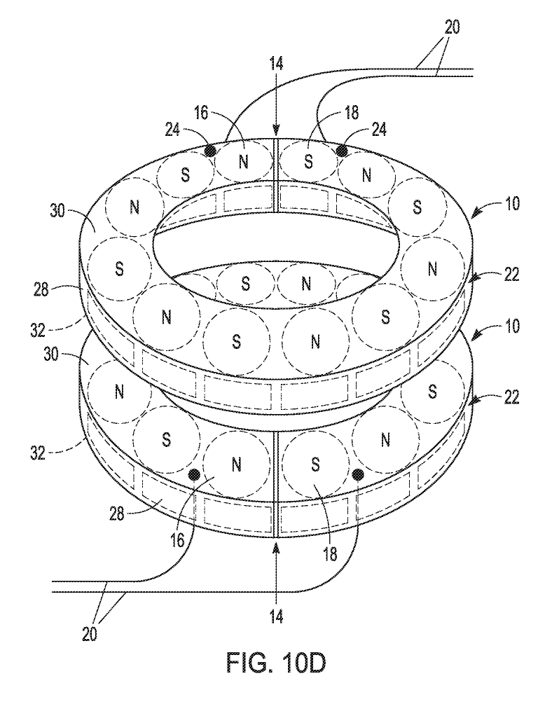

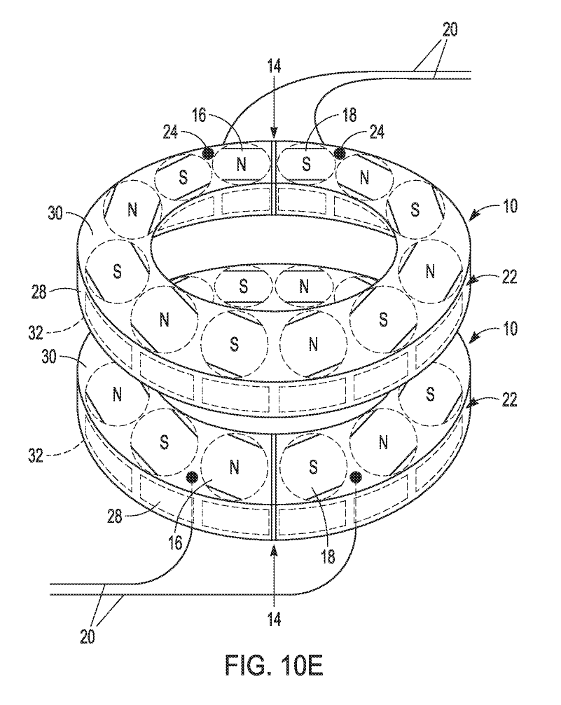

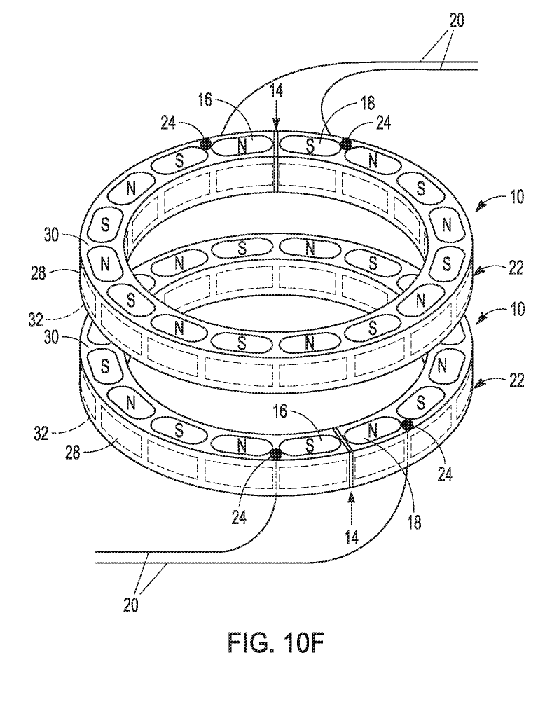

FIGS. 9 and 10A-F illustrate detailed MAD design examples such as magnet arrangement in the assembly, MAD ring-to-ring interaction. As discussed previously, there are many ways to design and make the MAD in terms of magnet geometric shape, dimensions, magnetic material type and their force and strength, their pole direction arrangement in assembly, the way to fix the magnets in the device, and the combination and arrangement to achieve a MAD with the lowest profile and strongest magnetic force. Disc magnets were used in the following examples to illustrate the options of their arrangement; however, embodiments of the present invention are not limited to disc magnets. The MAD is in linear configuration during delivery and reshaped to ring configuration after deployment; one way to have a ring formed inside of body lumen is to use the guiding elements to pull the distal and proximal segments together. In some embodiments, the magnet end pieces have opposite magnetic pole direction so that the attractive force between end pieces preserves the circularized ring shape during manipulations to mate. As illustrated in FIG. 9, in which the magnet pole direction is arranged such that north and south poles are alternated to each other in the S-N arrangement. With this arrangement, no special end piece arrangement is required; as long as total number of discs in a ring is an even number, the end pieces are always in S-N arrangement that can connect to each other. The round, oval, or oblong disc shape has an advantage of their rounded surface contact between each other, which acts as a hinge point during circling to form a ring. Due to the design of the MAD including magnet shape, pole direction in the arrangement and uniform heat shrink sheath wrapping, the invented MAD ring has the characteristics of double side (face) mating capability (no orientation required) and self-alignment capability.

With the magnet arrangement illustrated in FIG. 10A, the resulting MAD device rings have selective attraction faces, e.g., only S-side and N-side of the ring facing each other attracts (see FIG. 10A) and otherwise, N-side to N-side or S-side to S-side repels each other (e.g., one of the ring plane has to be turned 180° in order to get attraction).

With the magnet arrangement illustrated in FIG. 9, the resulting MAD device has no orientation requirement to mate or attract each other and the S and N poles of the magnets will align vertically for attraction automatically when two rings approach one another with the pole directions indicated (self-alignment), as shown in 10B, showing guiding lines attached to the end magnets via eyelet loops. Another illustration is shown in 10C, with the guiding lines directly attached to the heat shrink tube, through the tube with the knots on the top and bottom surfaces. In another embodiment, a single guiding element line may pass through the heat-shrink tube of the proximal magnet and be attached with a breakaway knot to the heat-shrink tube at the distal end, so that as it is retracted it pulls proximal and distal ends together to circularize the magnet ring.

With the magnet arrangement illustrated in FIG. 10D, the resulting MAD device rings have a smaller passing profile when in a linear configuration. In the linear arrangement, the chamfer features of the discs are aligned together along the longitudinal direction. The magnetic pole directions are indicated with S and N between two facing surfaces. The guiding lines directly attach to the heat shrink tube, through the tube with the knots on the top surfaces and the lines exit at another surfaces or vice versa. Another illustration is given in FIG. 10E, having the guiding lines directly attached to the heat shrink tube, half way through the tube with the knots on the top surfaces and the lines exiting at the middle of the side walls. The knot can be at bottom surface as well. Another example in FIG. 10F shows the least profile arrangement of oblong discs in a MAD assembly, in which the long axis of the discs are aligned with the ring circumference direction. The guiding element lines are directly connected to the heat shrink tube with knots being at top or bottom surface and the lines exiting from the middle side wall.

As shown in FIGS. 10A-F, the magnets in the rings have to match their pole direction (e.g., S-N or N-S) in order to attract each other between two rings. One of the rings will have to rotate a certain amount to achieve this polarity alignment with the other ring, typically the rotation angle is less than 45 degrees, preferably less than 25 degrees. The possible rotation angle of the MAD ring is dependent upon MAD mobility in the intestine and any restriction from the delivery system. The maximum degrees of rotation angle is to move one disc length radians, therefore, the number of discs in the ring is very important for mating ability. In one exemplary embodiment, to meet less than 25 degrees rotation angle, the nearest number of discs in a ring is 16 (the number has to be even). So any numbers at 16 or higher meets the above preferred characteristic regardless of geometric shape of the disc.

In various embodiments, the present invention provides a method for delivering the MAD to a body lumen, including two endoscopes advancing to the target site. For example, the method can include inserting an endoscope and advancing it to the target site in the first body lumen from one direction, and inserting second endoscope and advancing to the target site in the second body lumen from another direction. In some cases, the tips of the two endoscopes come into close proximity during advancement or manipulation. Then, two scope tips are adjacent to each other, the MADs are delivered from both direction and mated. However, in other cases, it is very challenging to bring tips into close proximity during advancement. But as the endoscopes are advanced, the scope shaft loops of the two endoscopes press against each other, and a point on the scope shafts may be identified at which the tips will be in proximity during withdrawal, which is an anatomically convenient site for mating. In various embodiments, the present invention provides a method for delivering the MAD to a body lumen, including two endoscopes advancing beyond the target site. For example, the method can include inserting an endoscope and advancing it beyond the target site in the first body lumen from one direction, and inserting second endoscope and advancing it beyond the target site in the second body lumen from another direction. An anatomically convenient mating site may then be identified as the point on the shafts at which the scope loops of the first and second endoscopes are proximate in two orthogonal planes by fluoroscopy. The MADs may then be delivered from both directions and mated after or while pulling back the endoscopes and the MADs to the desired target anastomosis site. This pull-back method has several advantages, as it precludes inadvertent capture of other tissues between mated magnets and shortens the procedure time. It also allows the primary manipulation of the rings to be drawing them back through the body lumen which makes control of the rings alignment easier than when trying to advance them forward down the body lumen.

In the embodiments of the present invention a method for delivering the MAD to a body lumen comprises 1) inserting and advancing the first endoscope with the first assisting devices transorally into stomach and duodenum to or beyond targeted site, such as jejunum; 2) inserting and advancing the second endoscope or colonoscope transanally into colon or ileum with the second assisting device to or beyond targeted site, such as ileum; 3) identifying the point on both endoscopes that are in close proximity; 4) delivering the MAD from the working channels of the endoscopes; 5) transforming the MAD from linear to circular shape; 6) manipulating or withdrawing the endoscopes to the previously identified point of proximity and mating two MAD rings with each other from two separated lumens/organs; 7) Using the tip of the delivery system to support the side of the MAD ring while pulling the guide element lines to separate the lines from the MAD rings, 8) withdrawing the MAD delivery systems into the endoscopes and withdrawing the endoscopes from the body; and 9) forming anastomosis over time. The first step and the second step can be performed at the same time. The first endoscope can be the same as or different from the second endoscope. The first assisting device can be the same as or different from the second assisting device. The assisting devices include overtube, single balloon overtube, double balloon overtube, spiral overtube, motorized spiral overtube, G-EYE endoscope system, and NaviAid balloon system. The first and the second assisting device can be NaviAid balloon system in one embodiment. The first assisting device is one of overtubes and the second assisting device is NaviAid balloon system in another embodiment. The pairs of the first and the second assisting devices are single balloon overtube and double balloon overtube, two overtubes, two double balloon systems, and two single balloon systems, one single balloon system and one double balloon system, two G-EYE endoscope systems, two NaviAid Balloon systems, and One G-EYE endoscope system and one NaviAid Balloon system. In various embodiments, both MAD catheters are advanced to the target site in the body lumens prior to releasing either MAD into the body lumen. In various embodiments, both MAD catheters are advanced beyond the target mating site prior to releasing either MAD into the body lumen and the MADs are pulled back rather than advanced to the mating site. The magnetic anastomosis device can include: at least two, three, four, five, six, seven, eight, nine, ten, twelve, fourteen, or sixteen magnets; and a flexible polymeric tube, wherein a plurality of the magnets are assembled and fixed in right direction and right place (e.g., has a desired orientation and a desired location) in the polymeric tube upon heating; and wherein the polymeric tube is flexible and conformable to allow the MAD to form into a ring shape.

In the embodiments of the present invention the methods to treat the diseases of diabetes and obesity comprises 1) inserting and advancing the first endoscope with the first assisting devices transorally into stomach and duodenum to or beyond targeted site, such as jejunum; 2) inserting and advancing the second endoscope or colonoscope transanally into colon or ileum with the second assisting device to or beyond targeted site, such as ileum; 3) identifying the point on both endoscopes that are in close proximity; 4) delivering the MAD from the working channels of the endoscopes; 5) transforming the MAD from linear to circular shape; 6) manipulating or withdrawing the endoscopes to the previously identified point of proximity and mating two MAD rings with each other from two separated lumens/organs; 7) Using the tip of the delivery system to support the side of the MAD ring while pulling the guide element lines to separate the lines from the MAD rings, 8) withdrawing the MAD delivery systems into the endoscopes and withdrawing the endoscopes from the body; 9) forming anastomosis over time; and 10) reducing Glycated Hemoglobin A1c(HGBA1C or HbA1c or A1C), plasma glucose concentration and body weight over time. The first step and the second step can be performed at the same time. The first endoscope can be the same as or different from the second endoscope. The first assisting device can be the same as or different from the second assisting device. The assisting devices include overtube, single balloon overtube, double balloon overtube, spiral overtube, motorized spiral overtube, G-EYE endoscope system, and NaviAid balloon system. The first and the second assisting device can be NaviAid balloon system in one embodiment. The first assisting device is one of overtubes and the second assisting device is NaviAid balloon system in another embodiment. The pairs of the first and the second assisting devices are single balloon overtube and double balloon overtube, two overtubes, two double balloon systems, and two single balloon systems, one single balloon system and one double balloon system, two G-EYE endoscope systems, two NaviAid Balloon systems, and One G-EYE endoscope system and one NaviAid Balloon system. The Glycated

Hemoglobin A1c (HGBA1C or HbA1c or A1C) can be reduced in the range of 0.1 to 4%, 0.5 to 3%, or 1 to 2%. The plasma glucose concentration can be reduced in the range of 200 to 1 mg/dl, 150 to 50 mg/dl, or 100 to 50 mg/dl. The body weight can be reduced in the range of 1% to 50%, 5% to 25%, or 10% to 15%. In one embodiment, the diseases for this treatment include one of diabetes, obesity, nonalcoholic fatty liver disease, digestive diseases, cancers, tumors. The tissues applicable to such a treatment include nonvascular lumens, digestive lumens, duodenum, jejunum, ileum, colon, cancers, and tumors.

Various embodiments provide a method for delivery of the MAD to a body lumen, the method including: inserting a MAD catheter and advancing to the target site in the body lumen; releasing the MAD to the body lumen; inserting the second MAD catheter and advancing to the target site to be joined in a body lumen; releasing the second MAD to the body lumen; manipulating guiding elements to align and place both of the MADs in a location and orientation to couple; mating the two MADs; removing the guiding elements from the MADs; withdrawing the MAD delivery systems from the body lumen. In various embodiments, both MAD catheters are advanced to the target site in the body lumens prior to releasing either MAD into the body lumen. In various embodiments, both MAD catheters are advanced beyond the target mating site prior to releasing either MAD into the body lumen and the MADs are pulled back rather than advanced to the mating site. The magnetic anastomosis device can include: at least two, three, four, five, six, seven, eight, nine, ten, twelve, fourteen, or sixteen magnets; and a flexible polymeric tube, wherein a plurality of the magnets are assembled and fixed in right direction and right place (e.g., has a desired orientation and a desired location) in the polymeric tube upon heating; and wherein the polymeric tube is flexible and conformable to allow the MAD to form into a ring shape.

In one embodiment, the method includes: inserting a MAD catheter and advancing to the target site in the body lumen; inserting the second MAD catheter and advancing to the target site to be joined in a body lumen and passing the tip of the first MAD catheter by at least about 5 to 10 cm; shaping the MADs into rings by pulling guiding elements; using pull-back method and manipulating guiding elements and catheters to orientate and place both of the MAD in a location and orientation to mate; after mated, detaching the first MAD to the body lumen; detaching the second MAD to the body lumen; withdrawing the MAD delivery systems from the body lumen.

In another embodiment, the endoscope can reach deep in the duodenum, jejunum, ileum or colon with the assistance of an endoscopic balloon catheter, such as NaviAid Balloon catheters from Smart Medical Systems Ltd., Israel. The role of the balloon catheter is to facilitate advancement of a standard endoscope into the small and large intestine, duodenum, jejunum, ileum, and colon. During the procedure of delivering the MADs in the body lumen, the endoscope can be inserted orally into duodenum, then the balloon catheter NaviAid AB (40 mm balloon) can be delivered thru the working channel of endoscope and advances beyond the scope distal tip further down to the body lumen. The balloon is inflated to fix the catheter at the position as an anchoring device for endoscope to advance, for example, beyond the ligament of Treitz. Optionally, the scope can be pulled back to reduce the loops in stomach and then moved forward by riding over the balloon catheter and reaching the anchoring balloon; then the balloon can be deflated and moved further down if needed and the above steps can be repeated to advance the scope to or beyond the targeted site, for example, in jejunum. Alternatively, the scope can follow the uninflated balloon as would be done with a guidewire. For the second MAD delivery, endoscope/colonoscope can be inserted into the body transanally inside of colon until reaching the ileocecal valve or passing just through the ileocecal valve. When the scope tip is at the ileocecal valve, the NaviAid AB can be inserted and passed through the valve into the ileum lumen. The AB balloon can be inflated and the scope advanced, riding over the balloon catheter shaft until seeing the balloon in front. If further advancement is required, the AB balloon can be deflated and moved forward to deeper location in the ileum, then inflated again and the scope moves further over the shaft until reaching targeted site, with repetition as needed. Alternatively, the scope can follow the uninflated balloon as would be done with a guidewire. Once both of the scopes entering from mouth and anus reach at or beyond the targeted site, the point where the scopes are in close proximity (the anatomically convenient target site) is identified by orthogonal fluoroscopic views. In some cases, this may be the tip of the scope; in others it may be a point on the shaft. In some embodiments, it is preferable that the scope tips are cross passing each other for at least 5 to 10 cm. Once the anatomically convenient target site has been identified, the balloon catheters can be withdrawn from the scopes, and then MAD systems can be delivered as follows: 1) the MADs can be delivered through the working channels of the endoscopes in a linear form; 2) the MAD can be transformed from linear to circular shape; 3) two MAD rings can mate each other from two separated lumens/organs via withdrawal of the two MAD rings and endoscopes to the previously identified point of proximity; 4) two MAD rings are detached from the guiding element; 5) the MAD delivery systems can be withdrawn from the endoscopes; 6) side-by-side jejunum and ileum anastomosis can be formed over time.

In another embodiment, the endoscope can reach deep in the duodenum, jejunum, ileum or colon with the assistance of an ancillary endoscope advancement device, such as NaviAid Balloon catheter (Smart Medical Systems Ltd., Israel), Spiral enteroscopy overtube (Olympus Medical, Japan), Single balloon overtube (Olympus Medical, Japan), Double balloon enteroscopy system (Fuji Medical, Japan), G-EYE (Smart Medical Systems). The role of the ancillary device can include facilitating advancement of a standard endoscope into the small and large intestine. Other techniques and tools for facilitating deep enteroscopy may be utilized to advance the endoscopes to or beyond the desired target site for anastomosis.

In various embodiments, the methods to treat the diseases of diabetes and obesity include (1) advancing endoscopes (concurrently or sequentially) orally into the jejunum and anally into the ileum beyond the intended depth for anastomosis (2) examining by fluoroscopy the scope loop patterns in two orthogonal planes or by rotation through orthogonal planes (3) identifying a point on each endoscope shaft that is in close proximity (for example, less than 2 cm or preferably less than 1 cm) to or directly contacting the other endoscope shaft (the target site); (4) deploying magnetic anastomosis devices concurrently or sequentially; (5) retracting the endoscopes to bring the MAD devices to the previously identified target site; (6) manipulating the MAD devices to mate to create a compression anastomosis.

In some embodiments, method of treating diabetes or obesity includes (1) advancing endoscopes orally into the jejunum and anally into the ileum beyond the intended depth for anastomosis (2) examining by fluoroscopy the scope loop patterns in two orthogonal planes or by rotation through orthogonal planes (3) identifying a point on each endoscope shaft that is in close proximity to or directly contacting the other endoscope shaft (the target site); (4) retracting the endoscopes to bring the MAD devices near the previously identified target site; (5) deploying MAD devices concurrently or sequentially; (6) manipulating the MAD devices to mate to create a compression anastomosis.

The step in some embodiments of advancing the endoscope deeper than the intended mating site and identifying and pulling back to a location where both endoscopic shafts are already in close proximity confers several novel advantages over methods disclosed in the prior art. First, the most anatomically convenient target site for mating is identified in advance. Second, it precludes capture of undesired tissue (additional to the intended intestinal walls) between the magnets, which might otherwise be captured if the scope tips are instead pushed into proximity. In addition, it precludes prolapse of the magnets prior to mating and minimizes any potential injury to wall tissue or perforation that might occur from advancement with the push tube out front. The result is a significantly shorter procedure time with a lower radiation dose. For example, without this procedural step, attempted mating of prior art magnets (as described in US 2016/0262761, the contents of which are hereby incorporated by reference) required a prolonged endoscopic procedure time of 115 minutes and laparoscopic assistance for mating in 8 of 10 of cases (As described in Machytka et al. Gastrointestinal Endoscopy 2017 pii: S0016-5107(17)32090-4 as described in US 20160262761, the contents of which are hereby incorporated by reference).

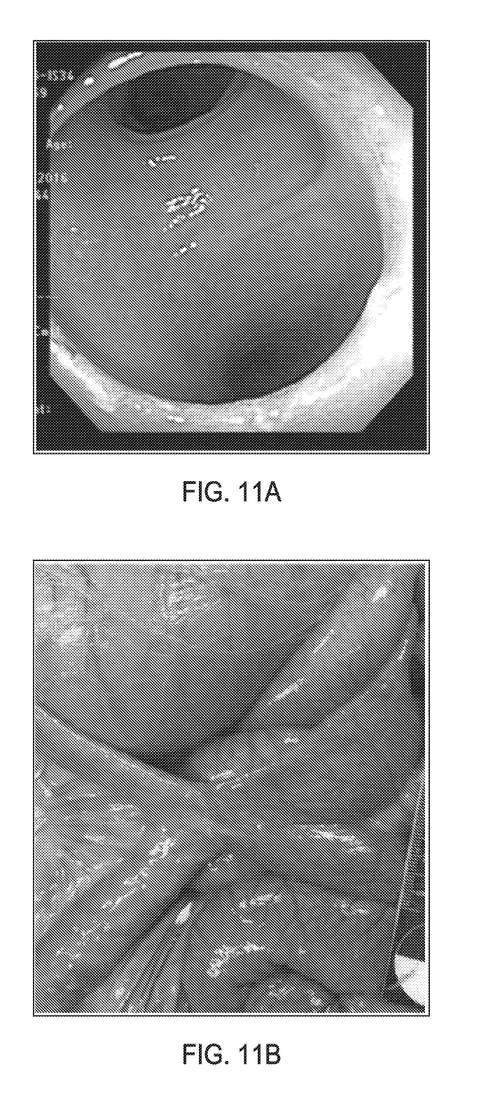

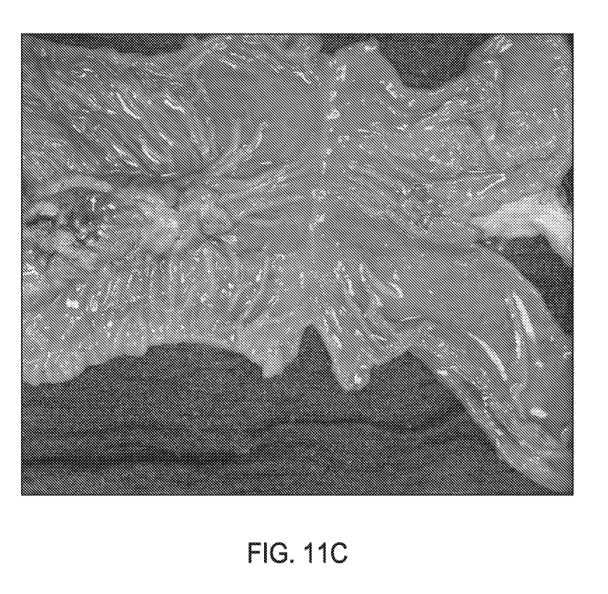

Pre-clinical results of swine are exhibited in FIGS. 11A-C with disc shape MADs. The magnets were arranged in the heat shrink tube with the alternated magnetic pole direction at one plane, e.g., -N-S-N-S-N-S-arrangement. The magnets were coated with Ni-Cu-Ni to seal the magnetic material and with Au as a top finishing coating for biocompatibility. Other metal or polymer coatings or the combinations thereof, described herein, can yield the same or similar biocompatibility. The outside diameter of MAD ring was about 20 mm. Two MADs were placed at two separate locations of small intestine and these two segments of small intestine were brought together by the two MAD devices (rings). All pictures were taken at two weeks' time point after the MADs implanted. FIG. 11A was taken with endoscope showing anastomosis and smooth connecting tissue surface from inside, which had normal wall thickness as the native intestine wall thickness. External picture of small intestine anastomosis is shown in FIG. 11B. It shows normal tissue structure, blood vessels and surface texture at anastomosis site. The connection between two segments of the intestine is smooth, sealed and completely healed.

Close exam with cut and opened intestine at another anastomosis site 90 days after MAD implant further confirmed these conclusions (see FIG. 11C). The connection line is strong and durable; the strength is equal or better than the native intestine wall and sometimes the connection line is stronger than the native intestine wall (as described in Ramin Jamshidi et al., J. of Pediatric Surgery (2009) Vol. 44, 222-228, the contents of which are hereby incorporated by reference).

### Additional Embodiments

The following exemplary embodiments are provided, the numbering of which is not to be construed as designating levels of importance:

Embodiment 1 provides a magnetic anastomosis device (MAD) comprising:
at least twelve magnets;
a tube comprising the at least twelve magnets;
wherein the tube is flexible and conformable so that the MAD transforms its shape from a ring shape to a linear shape to ring shape.

Embodiment 2 provides the MAD of Embodiment 1, wherein the tube is a polymer heat shrink tube (PHST).

Embodiment 3 provides the MAD of any one of Embodiments 1-2, wherein the tube is heated with the at least four magnets suitably positioned therein to form the MAD.

Embodiment 4 provides the MAD of any one of Embodiments 1-3, wherein the tube is durable to protect the magnet, a coating thereof, or a combination thereof, from damage.

Embodiment 5 provides the MAD of any one of Embodiments 1-4, wherein the tube acts as a protector for brittle and fragile neodymium magnets.

Embodiment 6 provides the MAD of any one of Embodiments 1-5, wherein guiding elements attach to the periphery of the at least one magnet and inside of the tube.

Embodiment 7 provides the MADs of Embodiment 6, wherein the guiding elements attach to the at least one magnet directly.

Embodiment 8 provides the MADs of any one of Embodiments 6-7, wherein the at least one magnet directly connected to the guiding elements comprises two open end magnets in the MAD, the two open end magnets each comprising an eyelet loop.

Embodiment 9 provides the MADs of any one of Embodiments 6-8, wherein the at least one magnet directly connected to guiding elements comprise two open end magnets in the MAD with an arc-shaped loop on the magnets.

Embodiment 10 provides the MAD of any one of Embodiments 6-9, wherein the guiding elements comprise single or multiple polymer filaments, sutures, braided lines, metal wires, or combinations thereof.

Embodiment 11 provides the MAD of any one of Embodiments 6-10, wherein the guiding elements comprise a polyester, a polyamide, a fluoropolymer, a polyethylene, a polypropylene, or a combination thereof.

Embodiment 12 provides the MAD of any one of Embodiments 6-11, wherein the number of the guiding elements is from 1 to 10.

Embodiment 13 provides the MAD of any one of Embodiments 1-12, wherein one or more guiding elements attach to the tube directly.

Embodiment 14 provides the MAD of Embodiment 13, wherein the guiding element attaches to the tube inside of the tube, outside of the tube, or a combination thereof.

Embodiment 15 provides the MAD of any one of Embodiments 13-14, wherein the guiding element attaches to the tube via one or more knots.

Embodiment 16 provides the MAD of Embodiment 15, wherein the one or more knots are located on top or bottom of tube surface, inside of tube surface, or a combination thereof.

Embodiment 17 provides the MAD of any one of Embodiments 15-16, wherein the guiding element exits the tube from an opposite surface from entry, in the middle of a side wall of the tube, or a combination thereof.

Embodiment 18 provides the MAD of any one of Embodiments 15-17, wherein the knot is located at a side wall of the tube.

Embodiment 19 provides the MAD of any one of Embodiments 15-18, wherein a ratio of the diameter of the knot to the hole diameter in the tube for passing the guiding element comprising the knot therethrough is from 1.1 to 2.0 and the knot is bigger than the hole.

Embodiment 20 provides the MAD of any one of Embodiments 15-19, wherein the knot pull-through force is 0.5 to 3 lbs.

Embodiment 21 provides the MAD of any one of Embodiments 1-20, wherein the tube comprises a polyester, a polyamide, a polyether-amide block copolymer, a polyolefin, a polyolefin derivative, polyvinyl chloride, a fluorinated ethylene propylene (FEP), a perfluoroalkoxy polymer (PFA), or a combination thereof.

Embodiment 22 provides the MAD of any one of Embodiments 1-21, wherein a size ratio between the circumference of the heat shrink tube inside diameter and the perimeter of the magnet C_{ID}/P_{cs} is in between 1 to 2.

Embodiment 23 provides the MAD of any one of Embodiments 1-22, wherein heat shrink tube is flexible, conformable, and durable, and has a wall thickness in the range of 0.15 mm to 0.50 mm.

Embodiment 24 provides the MAD of any one of Embodiments 1-23, wherein the heat shrink tube acts as an enclosure for at least four the magnets, provides protection to the at least four magnets or a coating thereon, or a combination thereof.

Embodiment 25 provides the MAD of any one of Embodiments 1-24, wherein the heat shrink tube has shape retention or at least partial shape retention features adapted to assist the transformation of linear form to ring shape.

Embodiment 26 provides the MAD of any one of Embodiments 1-25, wherein the at least one magnet comprises rare earth compounds, neodymium compounds, very high magnetic energy materials, N52, N42, and N38 grade neodymium materials, and combinations thereof.

Embodiment 27 provides the MAD of any one of Embodiments 1-26, wherein the at least one magnet comprises a noble element-modified neodymium compounds.

Embodiment 28 provides the MAD of any one of Embodiments 1-27, wherein the numbers of magnets are in a range of 14 -26.

Embodiment 29 provides the MAD of any one of Embodiments 1-28, wherein the at least one magnet has higher than 100 ° C. maximum operating temperature.

Embodiment 30 provides the MAD of any one of Embodiments 1-29, wherein the at least one magnet comprises a protective coating comprising zinc, nickel, nickel-copper-nickel, gold, silver, parylene, epoxy, polytetrafluoroethylene (PTFE), a fluoropolymer, or a combination thereof.

Embodiment 31 provides the MAD of Embodiment 30, wherein the protective coating has a thickness of about 0.1 μm to about 30 μm.

Embodiment 32 provides the MAD of any one of Embodiments 29-31, wherein the protective coating of each layer has a thickness of about 1 um to about 15 μm.

Embodiment 30 provides the MAD of any one of Embodiments 27-29, wherein the protective coating has a thickness of about 1 μm to about 15 μm.

Embodiment 33 provides the MAD of any one of Embodiments 29-32, wherein the protective coating comprises a dual-layer or triple-layer or higher-layer coating, with each layer on top of the last.

Embodiment 34 provides the MAD of any one of Embodiments 29-33, wherein the protective coating comprises a dual-layer coating with the first layer comprising Zn, Ni, Ni-Cu-Ni, or a combination thereof, and with the outer second layer comprising Ag, Au, parylene, or a combination thereof.

Embodiment 35 provides the MAD of any one of Embodiments 29-34, wherein the protective coating comprises a triple-layer coating with the first layer comprising Zn, Ni, Ni-Cu-Ni, or a combination thereof, the second layer comprises one or more noble metals, and with the outer third layer comprises PTFE, parylene, or a combination thereof.

Embodiment 36 provides the MAD of any one of Embodiments 1-35, wherein the at least one magnet comprises a plating or coating material comprising gold, silver, or a combination thereof.

Embodiment 37 provides the MAD of any one of Embodiments 1-36, wherein the at least one magnet comprises a polymer top coating comprising parylene, epoxy, polytetrafluoroethylene, or a combination thereof.

Embodiment 38 provides the MAD of any one of Embodiments 1-37, wherein the at least one magnet comprises a top coating comprising a metal and a polymer.

Embodiment 39 provides the MAD of any one of Embodiments 1-38, wherein at least one the magnet has a shape that is chosen from disc, cylinder, circle, oval, oblong, square, block, cube, hexagonal, octagonal, trapezoidal, chamfer featured, bevel featured and a combination thereof.

Embodiment 40 provides the MAD of any one of Embodiments 1-39, wherein the at least one magnet is a modified disc or cylinder having a profile that is approximately circular, oval, elongated ellipse, or a combination thereof, and comprising a chamfer, fillet, bevel, trapezoid, or combination thereof.

Embodiment 41 provides the MAD of any one of Embodiments 1-40, wherein the south and north poles of the at least one magnet are magnetized axially or at thickness direction.

Embodiment 42 provides the MAD of any one of Embodiments 1-40, wherein the at least one magnet is magnetized diametrically.

Embodiment 43 provides the MADs of any one of Embodiments 1-42, wherein the at least one magnet is at least four magnets, wherein the south and north pole of the at least four magnets are in alternate directions vertically to surface of the MAD.

Embodiment 44 provides the MAD of any one of Embodiments 1-42, wherein the at least one magnet is at least four magnets, wherein the south and north poles of the at least four magnets are in the alternate directions parallel to surface of the MAD.

Embodiment 45 provides the MAD of any one of Embodiments 1-44, wherein the MAD has two open ends; and wherein the magnet at both ends has opposite poles and the opposite poles are attractive to each other being able to form the ring structure by the guiding elements.

Embodiment 46 provides the MAD of any one of Embodiments 1-45, wherein the two end magnets have features for the attachment of guiding element.

Embodiment 47 provides the MAD of Embodiment 46, wherein the features on the two end magnets include partial grooves on the side wall of the magnet and hook ring with eyelet loop that snaps into the grooves.

Embodiment 48 provides the MAD of any one of Embodiments 46-47, wherein the features on the two end magnets include partial grooves on the side wall of the magnet and hook ring with arc-shaped loop that snaps into the grooves.

Embodiment 49 provides the MAD of any one of Embodiments 46-48, wherein a hook ring on the two end magnets is permanently fixed on to the magnet with adhesive.

Embodiment 50 provides the MAD of any one of Embodiments 46-49, wherein a hook ring on the two end magnets is made of metals, plastics, or combinations thereof.

Embodiment 51 provides the MAD of any one of Embodiments 1-50, wherein the MAD has two open ends and comprises at least four magnets; and wherein the magnet at both ends has opposite poles and the opposite poles are attractive to each other being able to form the ring structure by guiding elements that attach to the tube near two end magnets via knots.

Embodiment 52 provides the MAD of Embodiment 51, wherein the guiding elements are attached to the tube via knots at outside surface of the tube, inside surface of the tube, or a combination thereof.

Embodiment 53 provides the MAD of any one of Embodiments 51-52, wherein the guiding elements are attached to the tube via knots at a top or bottom surface of the tube, at the side wall of the tube, or a combination thereof.

Embodiment 54 provides the MAD of any one of Embodiments 51-53, wherein the guiding elements are attached to the tube via knots at a top or bottom surface of the tube, at the side wall of the tube, or a combination thereof; wherein the guiding elements exit at opposite surface of the tube or at middle of the side wall of the tube.

Embodiment 55 provides the MAD of any one of Embodiments 51-54, wherein a ratio of the diameter of the knot to the diameter of the tube hole size is from 1.1 to 2.0.

Embodiment 56 provides the MAD of any one of Embodiments 1-55, wherein at least one magnet is cylinder- and/or disc-shaped and the outer diameter (D) of the MAD is equal to the product of number of the magnets (n) multiplied by the diameter (d) of the magnets divided by Pi and then adding the diameter (d) of the magnets, i.e., D is about equal to nd/π+d.

Embodiment 57 provides the MAD of any one of Embodiments 1-56, wherein the at least one magnet comprises a number of magnets in the range of 1-40.

Embodiment 58 provides the MAD of any one of Embodiments 1-57, wherein the at least one magnet comprises a number of magnets in the range of 14-26.

Embodiment 59 provides the MAD of any one of Embodiments 1-58, wherein the at least one magnet comprises a number of magnets in the range of 16-24.

Embodiment 60 provides the MAD of any one of Embodiments 1-59, wherein the at least one magnet comprises an even number of magnets.

Embodiment 61 provides the MAD of any one of Embodiments 1-60, wherein the at least one magnet comprises an odd number of magnets.

Embodiment 62 provides a MAD delivery catheter comprising:
an outer polymeric sheath;
a pushing tube comprising a polymeric outer layer and a PTFE or fluoropolymer inner layer;
at least one guiding element; and
a magnetic anastomosis device (MAD) comprising
   at least twelve magnets, and
   a flexible, conformable tube comprising the at least twelve magnets,
   wherein the tube is flexible and conformable so that the MAD forms a ring shape from a linear shape.

Embodiment 63 provides the MAD delivery catheter of Embodiment 62, wherein the pushing tube comprises an inner layer comprising a fluoropolymer.

Embodiment 64 provides a MAD delivery catheter comprising:
an outer polymeric sheath;
a pushing tube in the outer polymeric sheath that is longer than the outer polymeric sheath, the pushing tube comprising a distal end and a proximal end;
guiding elements that are detachable from the MAD delivery catheter after deployment; and
a MAD assembly comprising
   at least four magnets,
   wherein the magnets are arranged in alternating pole direction that is in disc thickness direction and
   a tube comprising the at least four magnets,
   wherein the tube is flexible and conformable so that the MAD forms a ring shape from a linear shape;
wherein
   the MAD assembly is located at the distal end of the outer polymeric sheath and pushing tube, and
   the guiding elements are attached to the MAD, and extend from the distal end to the proximal end in the pushing tube.

Embodiment 65 provides the MAD delivery catheter of Embodiment 64, wherein the outer polymeric sheath covers about 100% of all the magnets of the MAD assembly.

Embodiment 66 provides the MAD delivery catheter of any one of Embodiments 64-65, wherein the outer polymeric sheath is a short sheath or an introducer less than 6 inches.

Embodiment 67 provides the MAD delivery catheter of any one of Embodiments 64-66, wherein the outer polymeric sheath covers less than about 100% of all the magnets of the MAD assembly.

Embodiment 68 provides the MAD delivery catheter of any one of Embodiments 64-67, wherein the outer polymeric sheath covers less than 5 magnets of the MAD assembly.

Embodiment 69 provides the MAD delivery catheter of any one of Embodiments 64-68 wherein the outer polymeric sheath/introducer material has surface low friction feature.

Embodiment 70 provides the MAD delivery catheter of any one of Embodiments 64-69, wherein the pushing tube comprises multiple layers of material.

Embodiment 71 provides the MAD delivery catheter of any one of Embodiments 64-70, wherein the pushing tube comprises an inner layer comprising a fluoropolymer and an outer layer.

Embodiment 72 provides the MAD delivery catheter of any one of Embodiments 64-71, wherein the pushing tube comprises an extruded tube inserted into another separately extruded tube.

Embodiment 73 provides the MAD delivery catheter of any one of Embodiments 64-72, wherein the catheter tip is an atraumatic tip having shore

D hardness 55D or less.

Embodiment 74 provides the MAD delivery catheter of any one of Embodiments 64-73, wherein the MAD rings have double side mating property through two layers tissues.

Embodiment 75 provides the MAD delivery catheter of any one of Embodiments 64-74, wherein the MAD rings have the characteristic of self-alignment to align their discs' S-pole to N-pole between the mating rings to have good compression pressure on the tissues.

Embodiment 76 provides the MAD delivery catheter of any one of Embodiments 64-75, wherein the guiding element is removable and detachable by breaking away from MAD at a connection point by pulling a knot in the guiding element.

Embodiment 77 provides the MAD delivery catheter of any one of Embodiments 64-76, wherein the guiding element is detached by rolling away the line from the MAD at a connection point.

Embodiment 78 provides the MAD delivery catheter of any one of Embodiments 64-77, wherein the guiding element is detached by cutting the line at a connection point.

Embodiment 79 provides a method for delivery of MAD in body lumen or organ, the method comprising:
a) inserting a MAD catheter and advancing to or beyond the target site in a body lumen;
b) releasing the MAD to the body lumen;
c) inserting the second MAD catheter and advancing to or beyond the target site in a body lumen;
d) releasing the second MAD to the body lumen;
e) manipulating endoscopes with pull-back method to orientate and place both of the MADs in right site and the right direction and to mate;
f) detaching or/and withdrawing the guiding elements; and
g) withdrawing the MADs delivery systems from the body lumen.

Embodiment 80 provides the method according to Embodiment 79, wherein the MAD comprises:
at least twelve magnets; and
a tube comprising the at least twelve magnets;
wherein
   the tube is flexible and conformable so that the MAD forms a ring shape from a linear shape, and
   the at least one magnet is assembled and fixed in a desired orientation and location in the polymeric tube upon heating.

Embodiment 81 provides the method according to any one of Embodiments 79-80, wherein the body lumen is chosen from digestive lumens, duodenum, jejunum, ileum, colon, and a combination thereof.

Embodiment 82 provides a method for treatment of a disease, the method comprising:
inserting and advancing the first endoscope with the first assisting devices transorally into stomach and duodenum to or beyond targeted site, such as jejunum;
inserting and advancing the second endoscope or colonoscope transanally into colon or ileum with the second assisting device to or beyond targeted site, such as ileum;
identifying the point on both endoscopes that are in close proximity;
delivering the MAD from the working channels of the endoscopes;
transforming the MAD from linear to circular shape;
manipulating or withdrawing the endoscopes to the previously identified point of proximity and mating two MAD rings with each other from two separated lumens/organs;
using the tip of the delivery system to support the side of the MAD ring while pulling the guide element lines to separate the lines from the MAD rings,
withdrawing the MAD delivery systems into the endoscopes and withdrawing the endoscopes from the body;
forming anastomosis over time; and
reducing Glycated Hemoglobin A1c(HGBA1C or HbA1c or A1C), plasma glucose concentration and body weight over time.

Embodiment 83 provides the method according to the Embodiments 82, wherein the first endoscope is different from the second endoscope.

Embodiment 84 provides the method according to any one of Embodiments 82-83, wherein the assisting devices include overtube, single balloon overtube, double balloon overtube, spiral overtube, motorized spiral overtube, G-EYE endoscope system, and NaviAid balloon system.

Embodiment 85 provides a method for treatment of a disease, the method comprising:
inserting an access device comprising an endoscope, overtube, laparoscope, or a combination thereof orally to the target site in body lumen;
inserting MAD catheter into the access device and advancing to the target site;
inserting a second access device comprising an endoscope, overtube, colonoscope, laparoscope, or a combination thereof rectally to the target site in a different body lumen to be joined;
inserting the second MAD catheter into the second access device and advancing to the target site;
confirming the two access devices are close enough or touching each other for MAD to mate and advancing two scopes beyond the mating site at least 5 cm to 10 cm for ready to deploy;
pushing the pushing tube to release the first MAD to the body lumen, circularizing;
pushing the pushing tube to release the second MAD to the second body lumen, circularizing;
manipulating the scopes with pull-back method to orientate and place both of the MADs in a location and orientation to couple;
detaching and/or withdrawing the guiding elements;
withdrawing the MAD delivery catheters and the access devices from the body;
(allowing the tissues between the MADs to necrose and degenerate over time;
forming the anastomosis;
allowing body fluids together with two MADs to flow out of the body, optionally the two MADs can be removed from the body lumen endoscopically or laparoscopically;
reducing Glycated Hemoglobin A1c, plasma glucose concentration and body weight over time.

Embodiment 86 provides the method according to any one of Embodiments 81-85, wherein the MAD comprises:
at least twelve magnets; and
a flexible polymeric tube;
wherein
   the at least one magnet is fixed in a desired orientation and location in the polymeric tube upon heating, and
   the polymeric tube is flexible and conformable to allow the MAD to direct into a geometric shape.

Embodiment 87 provides the method according to any one of Embodiments 81-86 wherein the body lumen is chosen from nonvascular lumens, digestive lumens, duodenum, jejunum, ileum, cecum, colon, cancers, tumors, and a combination thereof.

Embodiment 88 provides the method according to any one of Embodiments 81-87, wherein the disease is chosen from diabetes, obesity, nonalcoholic fatty liver disease, digestive diseases, cancers, tumors, and a combination thereof.

Embodiment 89 provides a method for treatment of a disease, the method comprising:
placing a first MAD device in a first body lumen via transoral insertion;
placing a second MAD device in a second body lumen adjacent to the first body lumen via transanal insertion;
wherein the placing of the first MAD device and the second MAD device independently comprises
   placing the MAD device in the body lumen using an access device, or
   placing the MAD device in the body lumen using an access device.

Embodiment 90 provides the method for treatment of a disease of any one of Embodiments 87-88, wherein:
the first MAD device is placed in the first body lumen via transoral insertion using a scope, and
   the second MAD device is placed in the second body lumen via transanal insertion using a second scope, and
a pull-back method is used to mate MADs.

Embodiment 91 provides a method for treatment of a disease, the method comprising:
(1) inserting an endoscope orally and advancing beyond the target site in body lumen;
(2) inserting MAD catheter into the endoscope;
(3) inserting a second endoscope or colonoscope rectally beyond the target site in a body lumen to be joined;
(4) inserting a second MAD catheter into the second endoscope or colonoscope;
(5) pushing a first pushing tube to release the first MAD to the first body lumen, re-circularizing;
(6) pushing a second pushing tube to release the second MAD to the second body lumen, re-circularizing;
(8) pulling back the first endoscope and second endoscope or colonoscope while manipulating the guiding elements to orientate and place both of the MADs in a location and orientation to mate near the target site;
(9) cutting and/or withdrawing the guiding elements;
(10) withdrawing the MAD delivery catheters and the first endoscope and second endoscope or colonoscope;
(11) allowing the tissues between the MADs to necrotize and degenerate over time;
(12) forming the anastomosis; and
(13) allowing body fluids together with two MADs to flow out of the body, optionally the two MADs can be removed from the body lumen endoscopically or laparoscopically.

Embodiment 92 provides a method for treatment of a disease, the method comprising:
(1) inserting an endoscope orally and advancing beyond the target site in body lumen;
(2) inserting a second endoscope or colonoscope anally and advancing beyond the target site in a body lumen to be joined;
(3) confirming the target site may be reached by retraction of the two endoscopes;
(4) inserting MAD catheters into the first endoscope and second endoscope or colonoscope;
(5) pushing the pushing tubes sequentially or simultaneously to release the MADs into the body lumens to be joined;
(6) circularizing the MADs in both lumens using the guiding elements;
(7) manipulating the guiding elements to orientate and place both of the MAD in a location and orientation to couple during or after pullback of the endoscope;
(8) detaching and withdrawing the guiding elements;
(9) withdrawing the MAD delivery catheters and the first endoscope and second endoscope or colonoscope from the body;
(10) allowing the tissues between the MADs to necrotize and degenerate over time;
(11) forming the anastomosis; and
(12) allowing body fluids together with two MADs to flow out of the body, optionally the two MADs can be removed from the body lumen endoscopically or laparoscopically.

Embodiment 93 provides the method of Embodiment 92, wherein a balloon catheter is used to aid the advance of the endoscope to or beyond the target site for joining of lumens.

Embodiment 94 provides the MAD, MAD delivery catheter, or method of any one or any combination of Embodiments 1-93 optionally configured such that all elements or options recited are available to use or select from.

What is claimed is:

1. A magnetic anastomosis device (MAD) comprising:
at least twelve magnets;
a tube comprising the at least twelve magnets;
wherein the tube is flexible and conformable so that the MAD forms a ring shape from a linear shape.

2. The MAD of claim 1, wherein the tube is a polymer heat shrink tube (PHST).

3. The MAD of claim 1, wherein the tube is heated with the at least twelve magnets suitably positioned therein to form the MAD.

4. The MAD of claim 1, wherein the tube is durable to protect the magnets, a coating thereof, or a combination thereof, from damage.

5. The MAD of claim 1, wherein the tube acts as a protector for brittle and fragile neodymium magnets.

6. The MAD of claim 1, wherein guiding elements attach to the periphery of the at least one magnet and inside of the tube.

7. The MAD of claim 6, wherein the guiding elements attach to the at least one magnet directly.

8. The MAD of claim 6, wherein the at least one magnet directly connected to the guiding elements comprises two open end magnets in the MAD, the two open end magnets each comprising an eyelet loop.

9. The MAD of claim 6, wherein the at least one magnet directly connected to guiding elements comprise two open end magnets in the MAD with an arc-shaped loop on the magnets.

10. The MAD of claim 6, wherein the guiding elements comprise single or multiple polymer filaments, sutures, braided lines, metal wires, or combinations thereof.

11. The MAD of claim 6, wherein the guiding elements comprise a polyester, a polyamide, a fluoropolymer, a polyethylene, a polypropylene, or a combination thereof

12. The MAD of claim 6, wherein the number of the guiding elements is from 1 to 10.

13. The MAD of claim 1, wherein one or more guiding elements attach to the tube directly.

14. The MAD of claim 13, wherein the guiding element attaches to the tube inside of the tube, outside of the tube, or a combination thereof.

15. The MAD of claim 13, wherein the guiding element attaches to the tube via one or more knots.

16. The MAD of claim 15, wherein the one or more knots are located on top or bottom of tube surface, inside of tube surface, or a combination thereof.

17. The MAD of claim 15, wherein the guiding element exits the tube from an opposite surface from entry, in the middle of a side wall of the tube, or a combination thereof.

18. The MAD of claim 15, wherein the knot is located at a side wall of the tube.

19. The MAD of claim 15, wherein a ratio of the diameter of the knot to the hole diameter in the tube for passing the guiding element comprising the knot therethrough is from 1.1 to 2.0.

20. The MAD of claim 1, wherein the tube comprises a polyester, a polyamide, a polyether-amide block copolymer, a polyolefin, a polyolefin derivative, polyvinyl chloride, a fluorinated ethylene propylene (FEP), a perfluoroalkoxy polymer (PFA), or a combination thereof.

21. The MAD of claim 1, wherein a size ratio between the circumference of the heat shrink tube inside diameter and the perimeter of the magnet C_{ID}/P_{cs} is in between 1 to 2.

22. The MAD of claim 1, wherein heat shrink tube is flexible, conformable, and durable, and has a wall thickness in the range of 0.15 mm to 0.50 mm.

23. The MAD of claim 1, wherein the heat shrink tube acts as an enclosure for at least one the magnet, provides protection to the at least one magnet or a coating thereon, or a combination thereof.

24. The MAD of claim 1, wherein the heat shrink tube has at least partially shape retention capability such as MAD ring shape.

25. The MAD of claim 1, wherein the at least one magnet comprises rare earth compounds, neodymium compounds, very high magnetic energy materials, N52, N42, and N38 grade neodymium materials, and combinations thereof.

26. The MAD of claim 1, wherein the at least one magnet comprises a noble element-modified neodymium compounds.

27. The MAD of claim 1, wherein the at least one magnet has higher than 100° C. maximum operating temperature.

28. The MAD of claim 1, wherein the at least one magnet comprises a protective coating comprising zinc, nickel, nickel-copper-nickel, gold, silver, parylene, epoxy, polytetrafluoroethylene (PTFE), a fluoropolymer, or a combination thereof.

29. The MAD of claim 28, wherein the protective coating has a thickness of about 0.1 μm to about 30 μm.

30. The MAD of claim 28, wherein the protective coating has a thickness of about 0.1 μm to about 15 μm.

31. The MAD of claim 28, wherein the protective coating has a thickness of about 1 μm to about 15 μm.

32. The MAD of claim 28, wherein the protective coating comprises a dual-layer or triple-layer coating, with each layer on top of the last.

33. The MAD of claim 28, wherein the protective coating comprises a dual-layer coating with the first layer comprising Zn, Ni, Ni-Cu-Ni, or a combination thereof, and with the outer second layer comprising Ag, Au, parylene, or a combination thereof.

34. The MAD of claim 28, wherein the protective coating comprises a triple-layer coating with the first layer comprising Zn, Ni, Ni-Cu-Ni, or a combination thereof, the second layer comprises one or more noble metals, and with the outer third layer comprises PTFE, parylene, or a combination thereof.

35. The MAD of claim 1, wherein the at least one magnet comprises a plating or coating material comprising gold, silver, or a combination thereof.

36. The MAD of claim 1, wherein the at least one magnet comprises a polymer top coating comprising parylene, epoxy, polytetrafluoroethylene, or a combination thereof.

37. The MAD of claim 1, wherein the at least one magnet comprises a top coating comprising a metal and a polymer.

38. The MAD of claim 1, wherein the at least twelve magnets have a shape that is chosen from disc, cylinder, circle, oval, oblong, square, block, cube, hexagonal, octagonal, trapezoidal, and a combination thereof.

39. The MAD of claim 1, wherein the at least one magnet is a modified disc or cylinder having a profile that is approximately circular, oval, elongated ellipse, oblong or a combination thereof, and comprising a chamfer, fillet, bevel, trapezoid, or combination thereof.

40. The MAD of claim 1, wherein the magnets in the MAD assembly have round end like-half circle that acts as a hinge point between the discs for easier circling during the procedure.

41. The MAD of claim 1, wherein the south and north poles of the at least one magnet are magnetized axially or at thickness direction.

42. The MAD of claim 1, wherein the at least one magnet is magnetized diametrically.

43. The MAD of claim 1, wherein the at least one magnet is at least two magnets, wherein the south and north pole of the at least two magnets are in alternate directions vertically to surface of the MAD.

44. The MAD of claim 1, wherein the at least one magnet is at least two magnets, wherein the south and north poles of the at least two magnets are in the alternate directions parallel to surface of the MAD.

45. The MAD of claim 1, wherein the MAD has two ends; and wherein the magnets at both ends have opposite poles and the opposite poles are attractive to each other being able to form the ring structure by the guiding elements.

46. The MAD of claim 1, wherein the two end magnets have features for the attachment of guiding element.

47. The MAD of claim 46, wherein the features on the two end magnets include partial grooves on the side wall of the magnet and hook ring with eyelet loop that snaps into the grooves.

48. The MAD of claim 46, wherein the features on the two end magnets include partial grooves on the side wall of the magnet and hook ring with arc-shaped loop that snaps into the grooves.

49. The MAD of claim 46, wherein a hook ring on the two end magnets is permanently fixed on to the magnet with adhesive.

50. The MAD of claim 46, wherein a hook ring on the two end magnets is made of metals, plastics, or combinations thereof.

51. The MAD of claim 1, wherein the MAD has two open ends and comprises at least two magnets; and wherein the magnet at both ends has opposite poles and the opposite poles are attractive to each other being able to form the ring structure by guiding elements that attach to the tube near two end magnets via knots.

52. The MAD of claim 51, wherein the guiding elements are attached to the tube via knots at outside surface of the tube, inside surface of the tube, or a combination thereof.

53. The MAD of claim 51, wherein the guiding elements are attached to the tube via knots at a top or bottom surface of the tube, at the side wall of the tube, or a combination thereof.

54. The MAD of claim 51, wherein the guiding elements are attached to the tube via knots at a top or bottom surface of the tube, at the side wall of the tube, or a combination thereof; wherein the guiding elements exit at opposite surface of the tube or at middle of the side wall of the tube.

55. The MAD of claim 51, wherein a ratio of the diameter of the knot to the diameter of the tube hole size is from 1.1 to 2.0.

56. The MAD of claim 51, wherein pull through force for detachment is in a range of from about 0.5 to 3 lbs.

57. The MAD of claim 1, wherein at least one magnet is cylinder- and/or disc-shaped and the outer diameter (D) of the MAD is equal to the product of number of the magnets (n) multiplied by the diameter (d) of the magnets divided by Pi and then adding the diameter (d) of the magnets that is about equal to nd/π+d.

58. The MAD of claim 1, wherein the MAD ring linear profile is determined by the diameter (d) and thickness (h) of the discs; the ratio of d/h is 1.10 to 1.75 when the discs are elongated circle like oblongs, the ratio is width/thickness.

59. The MAD of claim 1, wherein the MAD ring linear profile can be improved significantly by applying chamfer features on to the round and oblong discs.

60. The MAD of claim 1, wherein the at least one magnet comprises a number of magnets in the range of 1-40.

61. The MAD of claim 1, wherein the at least one magnet comprises a number of magnets in the range of at least one of 8-30, 10-30, 12-30, 14-30, and 16-26.

62. The MAD of claim 1, wherein the at least one magnet comprises an even number of magnets.

63. The MAD of claim 1, wherein the at least one magnet comprises an odd number of magnets.

64. A MAD delivery catheter comprising:
an outer polymeric sheath;
a pushing tube comprising a polymeric outer layer and a PTFE or fluoropolymer inner layer;
at least one guiding element; and
a magnetic anastomosis device (MAD) comprising
at least twelve magnets, and
a flexible, conformable tube comprising the at least twelve magnets,
wherein the tube is flexible and conformable so that the MAD forms a ring shape from a linear shape.

65. The MAD delivery catheter of claim 60, wherein the pushing tube comprises an inner layer comprising a fluoropolymer.

66. A MAD delivery catheter comprising:
an outer polymeric sheath;
a pushing tube in the outer polymeric sheath that is longer than the outer polymeric sheath, the pushing tube comprising a distal end and a proximal end;
guiding elements that are detachable from the MAD delivery catheter after deployment; and
a MAD assembly comprising
at least twelve magnets, and
a tube comprising the at least twelve magnets,
wherein the tube is flexible and conformable so that the MAD forms a ring shape from a linear shape;
wherein
the MAD assembly is located at the distal end of the outer polymeric sheath and pushing tube, and
the guiding elements are attached to the MAD, and extend from the distal end to the proximal end in the pushing tube.

67. The MAD delivery catheter of claim 66, wherein the outer polymeric sheath covers about 100% of all the magnets of the MAD assembly.

68. The MAD delivery catheter of claim 66, wherein the outer polymeric sheath is a short sheath less than 6 inches.

69. The MAD delivery catheter of claim 66, wherein the outer polymeric sheath covers less than about 100% of all the magnets of the MAD assembly.

70. The MAD delivery catheter of claim 66, wherein the pushing tube comprises multiple layers of material.

71. The MAD delivery catheter of claim 66, wherein the pushing tube comprises an inner layer comprising a fluoropolymer and an outer layer.

72. The MAD delivery catheter of claim 66, wherein the pushing tube comprises an extruded tube inserted into another separately extruded tube.

73. The MAD delivery catheter of claim 66, wherein the catheter tip is an atraumatic tip having shore D hardness 55D or less.

74. The MAD delivery catheter of claim 66, wherein the guiding element is removable and detachable by breaking away from MAD at a connection point by pulling a knot in the guiding element through the tube.

75. The MAD delivery catheter of claim 66, wherein the guiding element is detached by rolling away the line from the MAD at a connection point.

76. The MAD delivery catheter of claim 66, wherein the guiding element is detached by cutting the line at a connection point.

77. The MAD delivery catheter of claim 66, wherein the MAD ring features double side mating property in which no ring face orientation is required to mate another ring.

78. The MAD delivery catheter of claim 66, wherein the MAD ring has self-alignment capability during MAD rings coupling to match N and S poles for attraction between the rings.

79. A method for the methods to treat the diseases of diabetes and obesity comprises
inserting and advancing the first endoscope with the first assisting devices transorally into stomach and duodenum to or beyond targeted site, such as jejunum;
inserting and advancing the second endoscope or colonoscope transanally into colon or ileum with the second assisting device to or beyond targeted site, such as ileum;
identifying the point on both endoscopes that are in close proximity;
delivering the MAD from the working channels of the endoscopes;
transforming the MAD from linear to circular shape;
manipulating or withdrawing the endoscopes to the previously identified point of proximity and mating two MAD rings with each other from two separated lumens/organs;
using the tip of the delivery system to support the side of the MAD ring while pulling the guide element lines to separate the lines from the MAD rings,
withdrawing the MAD delivery systems into the endoscopes and withdrawing the endoscopes from the body,
forming anastomosis over time, and
reducing Glycated Hemoglobin Al c(HGBA1C or HbA1c or A1C), plasma glucose concentration and body weight over time,
wherein the first endoscope is different from the second endoscope;
wherein the first assisting device can be the same or different from the second assisting device, wherein the assisting devices is one of overtube, single balloon overtube, double balloon overtube, spiral overtube, motorized spiral overtube, G-EYE endoscope system, and NaviAid balloon system.

80. The method according to claim 79, wherein the MAD comprises:
at least twelve magnets; and
a tube comprising the at least twelve magnets;
wherein
the tube is flexible and conformable so that the MAD forms a ring shape from a linear shape, and
the at least one magnet is assembled and fixed in a desired orientation and location in the polymeric tube upon heating.

81. The method according to claim 79, wherein the body lumen is chosen from nonvascular lumens, digestive lumens, duodenum, jejunum, ileum, cecum, colon, cancers, tumors, and a combination thereof.

82. The method of treating diabetes or obesity comprises:
advancing endoscopes orally into the jejunum and anally into the ileum beyond the intended depth for anastomosis;
examining by fluoroscopy the scope loop patterns in two orthogonal planes or by rotation through orthogonal planes;
identifying a point on each endoscope shaft that is in close proximity to or directly contacting the other endoscope shaft;
deploying magnetic anastomosis devices concurrently or sequentially;
retracting the endoscopes to bring the MAD devices to the previously identified target site; and
manipulating the MAD devices to mate to create a compression anastomosis.

83. The method of treating diabetes or obesity comprising:
advancing endoscopes orally into the jejunum and anally into the ileum beyond the intended depth for anastomosis;
examining by fluoroscopy the scope loop patterns in two orthogonal planes or by rotation through orthogonal planes
identifying a point on each scope shaft that is in close proximity to or directly contacting the other scope shaft;
retracting the endoscopes to bring the MAD devices to the previously identified target site;
deploying MAD devices concurrently or sequentially; and
manipulating the MAD devices to mate to create a compression anastomosis.
